(19) United States
(12) Patent Application Publication
(10) Pub. No.: US 2006/0189791 A1
(43) Pub. Date: Aug. 24, 2006

(54) Recombinant expression of streptococcus pyogenes cysteine protease and immunogenic compositions thereof

(75) Inventors: Laurie Anne Winter, Brockport, NY (US); Yury Vladimirovich Matsuka, New Windsor, NY (US); Elzabeth Teremy Anderson, Lansdale, PA (US); Stephen Bruce Olmsted, West Nyack, NY (US)

Correspondence Address: WYETH;PATENT LAW GROUP, 5 GIRALDA FARMS, MADISON, NJ, 07940, US

(73) Assignee: Wyeth Holdings corporation, Madison, NJ (US)

(21) Appl. No.: 10/564,264

(22) PCT Filed: Jul. 7, 2004
(86) PCT No.: PCT/US04/21714

§ 371 Date: Mar. 7, 2006

Related U.S. Application Data
(60) Provisional application No. 60/486,114, filed on Jul. 10, 2003.

(51) Int. Cl. C07K 14/315; C07H 21/04; C12P 21/06; C12N 1/21; C12N 15/74

(52) U.S. Cl. 530/350; 435/69.1; 435/320.1; 530/388.4; 536/23.7; 435/252.33

(57) ABSTRACT

The present invention generally relates to the fields of molecular biology, clinical bacteriology and protein folding. More particularly, the invention relates to methods for recombinantly expressing a soluble mature Streptococcus pyogenesexotoxin B (SpeB) polypeptide in a host cell.

### FIELD OF THE INVENTION

The present invention generally relates to the fields of molecular biology, clinical bacteriology and protein folding. More particularly, the invention relates to methods for recombinantly expressing a mature Streptococcus pyogenes exotoxin B (SpeB) polypeptide in a host cell.

### BACKGROUND OF THE INVENTION

Streptococcus pyogenes, also called group A streptococci (GAS), is a common gram-positive bacterial pathogen of humans. S. pyogenes causes a variety of conditions in humans including pharyngitis, impetigo and sepsis. Subsequent to infection, autoimmune complications such as rheumatic fever and acute glomerulonephritis also occur in humans. S. pyogenes also causes severe acute diseases such as scarlet fever, necrotizing fasciitis and toxic shock.

Sore throat caused by group A streptococci, commonly called “strep throat,” accounts for at least 16% of all office calls in a general medical practice, depending on the season (Hope-Simpson, 1981). Group A streptococci are also the cause of the recent resurgence in North America and four other continents of toxic shock associated with necrotizing fascilitis (Stevens, 1992).

Streptococcal infections are currently treated by antibiotic therapy. However, 25-30% of those treated have recurrent disease and/or shed the organism in mucosal secretions. Antibiotic treatment of toxic shock and severe invasive disease is frequently ineffective, and mortality can exceed 50% (Davies et al., 1996). The failure of penicillin to treat severe invasive streptococcal infections successfully is attributed to the phenomenon that a large inoculum reaches a stationary phase quickly and penicillin is not very effective against slow-growing bacteria (Stevens et al., 1993). Thus, there remains a continuing need for an effective means to prevent or ameliorate streptococcal infections. More specifically, a need exists to identify and develop antigens (or immunogens) useful in immunogenic compositions which prevent streptococcal infection. One such polypeptide antigen currently being considered as an immunogen is the S. pyogenes exotoxin B (SpeB), also known as streptococcal cysteine protease, streptococcal proteinase or streptopain.

S. pyogenes exotoxin B (SpeB) is expressed as a 40 kDa inactive pre-pro-enzyme (i.e., a zymogen) (Chaussee et al., 1993; Liu and Elliot., 1965), with a 27 amino acid NH₂-terminal signal sequence, followed by a 118 amino acid pro-peptide sequence (amino acids 28-145), and a 253 amino acid mature sequence (amino acids 146-398). Upon secretion, the 40 kDa SpeB zymogen undergoes autocatalytic activation resulting in the removal of the 12 kDa NH₂-terminal pro-peptide and formation of the mature, 28 kDa, active SpeB enzyme. This mechanism of conversion to active enzyme prevents unwanted protein degradation and enables spatial and temporal regulation of proteolytic activity (Khan and James, 1998).

As a member of the cysteine endopeptidase group of enzymes, SpeB contains a Cys-His pair at the active site (Liu et al., 1965; Liu, 1965; Tai et al., 1976). Replacement of the single cysteine residue at position 192 to serine (hereinafter, “C192S”) results in a loss of detectable proteolytic activity of the SpeB enzyme, preventing processing of the 40 kDa SpeB zymogen to the 28 kDa mature SpeB form (Gubba et al., 1998; Matsuka et al., 1999; Musser et a., 1996).

Results from previous studies have revealed that the mature form of the C192S SpeB mutant is required for the generation of antibodies with maximum inhibitory activity towards the wild-type SpeB enzyme (Matsuka et al., 1999). This suggests the need to produce the NH₂-terminally truncated, 28 kDa mature form of the C192S SpeB mutant for immunization purposes. However, recombinant expression of the mature C192S SpeB (i.e., lacking its NH₂-terminal pro-sequence) results in the accumulation of insoluble protein in E. coli.

One approach for producing soluble mature C192S SpeB mutant has been via limited proteolysis of the 40 kDa SpeB zymogen. For example, limited proteolysis of the 40 kDa C192S SpeB mutant zymogen to produce the mature C192S SpeB mutant has been achieved using several proteases including elastase, pepsin, thermolysin (Matsuka et al., 1999), and papain. These data, and data published by Liu and Elliot (1965) utilizing trypsin and subtilisin, suggest that the desired 28 kDa mature C192S SpeB mutant is successfully generated by treatment of the 40 kDa C192S SpeB mutant zymogen with a variety of proteinases.

However, this approach has several limitations for large-scale production. First, the final product yield of the mature protease is low due to the requirement for two successive purification steps, one for the full-length zymogen and the second for the processed mature protease. Secondly, there are difficulties associated with consistency and reproducibility of the limited proteolysis reaction, particularly on a larger scale. Lastly, there is an inherent risk of contamination of the final product with the enzymatically active exogenous protease used for cleavage. Such contamination is extremely difficult to avoid even when the reaction is carried out with resin-immobilized protease.

Thus, there remains a need in the art for immunogenic compositions effective against streptococcal infection in a mammalian host. It is therefore highly desirable to identify methods for producing or expressing the mature SpeB polypeptide, wherein the mature SpeB is immunogenic when administered to a mammalian host. It is also desirable that such methods for producing or expressing an immunogenic form of the mature SpeB polypeptide avoid the aforementioned large-scale limitations such as diminished SpeB yield, limited proteolysis consistency/reproducibility and the risk of exogenous enzyme contamination.

### SUMMARY OF THE INVENTION

The present invention broadly relates to methods for recombinantly expressing a mature Streptococcus pyogenes exotoxin B (SpeB) polypeptide in a host cell and immunogenic compositions thereof. More particularly, the invention is directed to novel methods for co-expressing the 12 KDa SpeB pro-peptide and the 28 kDa mature SpeB polypeptide in a host cell, wherein the mature SpeB polypeptide is soluble in the host cell.

Thus, in certain embodiments, the invention is directed to a method for recombinantly expressing a mature Streptococcus pyogenes exotoxin B (SpeB) polypeptide in a host cell, the method comprising (i) transforming, transducing, transfecting or infecting a host cell with a polycistronic plasmid, the polycistronic plasmid comprising (a) a polynucleotide sequence encoding a SpeB pro-polypeptide domain and (b) a polynucleotide sequence encoding a mature SpeB polypeptide, and (ii) culturing the host cell under conditions which permit the expression of the mature SpeB polypeptide and the SpeB pro-polypeptide domain by the host cell, and wherein the mature SpeB polypeptide is soluble in the host cell. Thus, in the polycistronic plasmid system of the present invention, a single promoter (e.g., a T7 promoter) drives the expression of a polycistronic mRNA transcript, wherein the polycistronic mRNA encodes two or more polypeptides in their correct reading frame (e.g., a SpeB pro-polypeptide domain and a mature SpeB polypeptide). In certain embodiments, the SpeB pro-polypeptide domain is further defined as a polypeptide comprising amino acid residues 28 through 145 of SEQ ID NO:2 and the mature SpeB polypeptide is further defined as a polypeptide comprising amino acid residues 146 through 398 of SEQ ID NO:2. In a preferred embodiment, the cysteine at amino acid residue 192 of the mature SpeB polypeptide is substituted by a serine. In another embodiment, the mature SpeB polypeptide is immunogenic in a mammalian host. In yet another embodiment, an antibody specific for the mature SpeB polypeptide cross-reacts with a wild-type SpeB polypeptide and neutralizes SpeB polypeptide activity. In certain preferred embodiments, the plasmid is a T7 promoter-containing plasmid. In one particular embodiment, the T7 promoter-containing plasmid is selected from the group consisting of pET, pRSET, pCRT7-CTTOPO and pIVeX. In another preferred embodiment, the host cell is a bacterial cell. In certain embodiments, the bacterial host cell is E. coli. In yet other embodiments, the E. coli is a strain selected from the group consisting of BLR(DE3), BLR(DE3)pLysS, AD494(DE3), AD494(DE3)pLysS, BL21(DE3), BL21(DE3) pLysS, BL21(DE3)pLysE, BL21 (DE3)pLacI, BL21trxB(DE3), BL21 trxB(DE3)pLysS, HMS174(DE3), HMS174(DE3)pLysS, HMS174(DE3)pLysE, Origami(DE3), Origami(DE3)pLysS, Origami(DE3)pLysE, Origami(DE3)pLacI, OrigamiB(DE3), OrigamiB(DE3)pLysS, OrigamiB(DE3)pLysE, OrigamiB(DE3)pLacI, Rosetta(DE3), Rosetta(DE3)pLysS, Rosetta(DE3)pLysE, Rosetta(DE3)pLacI, Tuner(DE3), Tuner(DE3)pLysS and Tuner(DE3)pLacI.

In other embodiments, the invention is directed to a method for recombinantly expressing a mature SpeB polypeptide in a host cell comprising (a) transforming, transducing, transfecting or infecting a host cell with (i) a plasmid comprising a polynucleotide sequence encoding a SpeB pro-polypeptide domain and (ii) a plasmid comprising a polynucleotide sequence encoding a mature SpeB polypeptide; and (b) culturing the host cell under conditions suitable to co-express the SpeB pro-polypeptide domain and the mature SpeB polypeptide, wherein the mature SpeB polypeptide is soluble in the host cell. In certain embodiments, the SpeB pro-polypeptide domain is further defined as a polypeptide comprising amino acid residues 28 through 145 of SEQ ID NO:2 and the mature SpeB polypeptide is further defined as a polypeptide comprising amino acid residues 146 through 398 of SEQ ID NO:2. In one preferred embodiment, the cysteine at amino acid residue 192 of the mature SpeB polypeptide is substituted by a serine. In other embodiments, the mature SpeB polypeptide is immunogenic in a mammalian host. In yet other embodiments, an antibody specific for the mature SpeB polypeptide cross-reacts with a wild-type SpeB polypeptide and neutralizes SpeB polypeptide activity. In another preferred embodiment, the plasmid is a T7 promoter-containing plasmid. In one particular embodiment, the T7 promoter-containing plasmid is selected from the group consisting of pET, pRSET, pCRT7-CTTOPO and pIVeX. In yet other embodiments, the host cell is a bacterial cell. In one preferred embodiment, the host cell is E. coli, wherein the E. coli is a strain selected from the group consisting of BLR(DE3), BLR(DE3)pLysS, AD494(DE3), AD494(DE3)pLysS, BL21(DE3), BL21(DE3) pLysS, BL21(DE3)pLysE, BL21(DE3)pLacI, BL21trxB(DE3), BL21trxB(DE3)pLysS, HMS174(DE3), HMS174(DE3)pLysS, HMS174(DE3)pLysE, Origami(DE3), Origami(DE3)pLysS, Origami(DE3)pLysE, Origami(DE3)pLacI, OrigamiB(DE3), OrigamiB(DE3)pLysS, OrigamiB(DE3)pLysE, OrigamiB(DE3)pLacI, Rosetta(DE3), Rosetta(DE3)pLysS, Rosetta(DE3)pLysE, Rosetta(DE3)pLacI, Tuner(DE3), Tuner(DE3)pLysS and Tuner(DE3)pLacI.

In another embodiment, the invention is directed to a method for producing a mature SpeB polypeptide comprising the steps of (a) recombinantly expressing in a host cell a plasmid comprising a polynucleotide sequence encoding a mature SpeB polypeptide, wherein the SpeB polypeptide forms an insoluble polypeptide aggregate in the host cell; (b) solubilizing the polypeptide aggregate, wherein the solubilized polypeptide is defined as a non-native mature SpeB polypeptide; (c) refolding the non-native mature SpeB polypeptide in the presence of a chaperone protein, wherein the non-native mature SpeB polypeptide is folded into a native mature SpeB polypeptide; and (d) recovering the native mature SpeB polypeptide. In a preferred embodiment, the chaperone protein is selected from the group consisting of GroEL, GroEUGroES, PDI, PPI and a SpeB pro-polypeptide domain. In a particular embodiment, the chaperone protein is a SpeB pro-polypeptide domain comprising amino acid residues 28 through 145 of SEQ ID NO:2. In a preferred embodiment, the mature SpeB is a polypeptide comprising amino acid residues 146 through 398 of SEQ ID NO:2. In another preferred embodiment, the cysteine at amino acid residue 192 of the mature SpeB polypeptide is substituted by a serine. In yet another embodiment, the insoluble polypeptide aggregate is further defined as an inclusion body. In other embodiments, solubilizing the polypeptide is a denaturant such as urea, guanidinium chloride, sodium dodecyl sulfate (SDS), heat and the like.

In still other embodiments, the invention is directed to a method for recombinantly expressing a mature SpeB polypeptide in a host cell comprising expressing in a host cell a polycistronic plasmid comprising (i) a polynucleotide sequence encoding a mature SpeB polypeptide and (ii) a polynucleotide sequence encoding a GroEL polypeptide, wherein the mature SpeB polypeptide is soluble in the host cell. In a preferred embodiment, the cysteine at amino acid residue 192 of the mature SpeB polypeptide is substituted by a serine. In a particular embodiment, the plasmid further comprises a polynucleotide encoding a GroES polypeptide.

In still other embodiments, the invention is directed to a method for producing a mature SpeB polypeptide comprising the steps of: (a) transforming, transducing, transfecting or infecting a host cell with a polycistronic plasmid comprising (i) a polynucleotide sequence encoding a mature SpeB polypeptide and (ii) a polynucleotide sequence encoding a GroEL polypeptide; (b) culturing the host cell under conditions suitable to express the mature SpeB polypeptide and the GroEL polypeptide, wherein the mature SpeB polypeptide is soluble in the host cell; and (c) recovering the native mature SpeB polypeptide. In a preferred embodiment, the cysteine at amino acid residue 192 of the mature SpeB polypeptide is substituted by a serine.

In certain other embodiments, the invention is directed to a mature SpeB polypeptide produced according to one or more of the methods set forth in the present invention. In other embodiments, the invention is directed to an immunogenic composition comprising a SpeB polypeptide produced according to one of the methods of the present invention. In still other embodiments, the invention is directed to a method of immunizing a mammalian subject against S. pyogenes, the method comprising administering to the subject an immunogenic amount of an immunogenic composition, wherein the immunogenic composition comprises a mature SpeB polypeptide produced according to the methods of the present invention.

In other embodiments, the invention is directed to a polycistronic plasmid comprising (a) a polynucleotide sequence encoding a SpeB pro-polypeptide domain and (b) a polynucleotide sequence encoding a mature SpeB polypeptide, wherein the mature SpeB polypeptide is soluble when expressed in a host cell. In certain embodiments, the cysteine at amino acid residue 192 of the mature SpeB polypeptide is substituted by a serine. In certain other embodiments, the plasmid is a T7 promoter-containing plasmid.

In certain other embodiments, the invention is directed to a plasmid comprising a polynucleotide sequence encoding (a) a SpeB pro-polypeptide domain and (b) a plasmid comprising a polynucleotide sequence encoding a mature SpeB polypeptide, wherein the mature SpeB polypeptide is soluble when expressed in a host cell. In preferred embodiments, the cysteine at amino acid residue 192 of the mature SpeB polypeptide is substituted by a serine. In other embodiments, the plasmid is a T7 promoter-containing plasmid.

In another embodiment, the invention is directed to a polycistronic plasmid comprising (a) a polynucleotide sequence encoding a mature SpeB polypeptide and (b) a polynucleotide sequence encoding a GroEL polypeptide, wherein the mature SpeB polypeptide is soluble when expressed in a host cell. In a preferred embodiment, the cysteine at amino acid residue 192 of the mature SpeB polypeptide is substituted by a serine. In certain other embodiments, the plasmid is a T7 promoter-containing plasmid.

In yet another embodiment, the invention is directed to a polycistronic plasmid comprising (a) a polynucleotide sequence encoding a mature SpeB polypeptide, (b) a polynucleotide sequence encoding a GroEL polypeptide and (c) a polynucleotide sequence encoding a GroES polypeptide, wherein the mature SpeB polypeptide is soluble when expressed in a host cell. In preferred embodiments, the cysteine at amino acid residue 192 of the mature SpeB polypeptide is substituted by a serine. In other embodiments, the plasmid is a T7 promoter-containing plasmid.

In still other embodiments, the invention is directed to a polycistronic plasmid comprising (a) a polynucleotide sequence encoding a mature SpeB polypeptide and (b) a polynucleotide sequence encoding one or more polypeptides selected from the group consisting of GroEL, GroES, PDI and PPI, wherein the mature SpeB polypeptide is soluble when expressed in a host cell. In a preferred embodiment, the cysteine at amino acid residue 192 of the mature SpeB polypeptide is substituted by a serine. In certain other embodiments, the plasmid is a T7 promoter-containing plasmid.

In another embodiment, the invention provides a host cell transformed, transduced, transfected or infected with a plasmid of the present invention.

Other features and advantages of the invention will be apparent from the following detailed description, from the preferred embodiments thereof, and from the claims.

### BRIEF DESCRIPTION OF THE FIGURES

FIGS. 1A and 1B demonstrate the interaction of recombinant mature SpeB and the pro-sequence domain. FIG. 1A depicts increasing concentrations of pepsin generated mature C192S SpeB (filled circles), and mature wild-type SpeB (filled squares), which were incubated in microtiter plates containing pro-sequence domain (filled symbols), or lysozyme (open symbols), and analyzed by ELISA as described in Example 1. Data are representative of three experiments, each performed in duplicate. FIG. 1B depicts real-time analysis of interactions between the pro-sequence domain and mature SpeB polypeptides, which was performed via use of a Biocore 3000 as described in Example 1. Results are expressed as surface plasmon resonance (relative response).

FIGS. 2A and 2B show pro-sequence domain mediated inhibition of mature SpeB. Depicted are inhibition of mature wild-type SpeB using resorufin-labeled casein (FIG. 2A) or cysteine protease papain (FIG. 2B) as a substrate, using the SpeB pro-sequence domain (filled squares), or lysozyme (open triangles) was analyzed as described in Example 1.

FIG. 3 shows the effect of recombinant pro-sequence domain on the refolding of denatured mature SpeB. Denatured mature SpeB was diluted rapidly in: PBS, 0.5 M arginine (filled circles); PBS, 0.5 M arginine, 20 μM protease inhibitor E-64 (open circles); PBS (filled squares) and PBS, 20 μM E-64 (open squares) containing increasing concentrations of pro-sequence domain. Reactions were performed as described in Example 1 and evaluated for the presence of protease activity using a resorufin-labeled casein cleavage assay.

FIG. 4 is a schematic representation of two-plasmid based expression constructs. Features of the two-plasmid based expression vectors include: KanR-kanamycin resistance gene (aph(3′)-Ia), AmpR-ampicillin resistance gene (β-lactamase), Ori-origin of replication, lacl^{q}-lac repressor, T7-based promoter (T7 or T7lac) and T7 terminator as indicated.

FIGS. 5A and 5B show a schematic of the polycistronic expression system and synthetic linker regions, respectively. Features of the polycistronic expression vector (FIG. 5A) include: KanR-kanamycin resistance gene (aph(3′)-la), Ori-origin of replication, lacl^{q}-lac repressor, T7lac promoter and T7 terminator as indicated. Base compositions of the linker regions analyzed are shown (FIG. 5B). Nucleotide designation (5 nt, 10 nt, 20 nt, 40 nt) of each linker region indicates the number of bases between the engineered TAA translational stop codon of the pro-sequence domain (PSD stop) (bold text), and optimized Shine-Dalgarno ribosome binding site (SD). The translational ATG start codon of the second cistron is shown in bold italic.

FIGS. 6A and 6B show an evaluation and relative quantitation of soluble mature SpeB and pro-sequence domain levels in polycistronic expression systems. An SDS-PAGE evaluation of the 5 nt, 10 nt, 20 nt and 40 nt linker-containing C192S SpeB polycistronic systems for expression of pro-sequence domain, and mature SpeB in soluble fractions was performed (FIG. 6A). Quantitation of expressed protein was accomplished by use of a Molecular Dynamics Personal Densitometer and measurement of area scanned denoted as level of expression (FIG. 6B).

FIG. 7 shows a quantitative PCR analysis of polycistronic cDNA. The cDNA and -RT controls were prepared and analyzed as described in Example 1. All Ct values were normalized to KanR mRNA expression.

FIG. 8 demonstrates the heat-induced denaturation of mature C192S SpeB. Denaturation curves for purified mature C192S SpeB generated by pepsin cleavage of expressed C192S SpeB zymogen, or by the two-plasmid and polycistronic co-expression systems were analyzed as described in Example 1.

FIG. 9 demonstrates the heat-induced denaturation of mature wild-type SpeB. Denaturation curves for purified mature wild-type SpeB produced by autocatalytic processing (i.e. zymogen generated), or by the two-plasmid and polycistronic co-expression systems were analyzed as described in Example 1.

FIG. 10 shows an evaluation of operational molarity for recombinant mature SpeB. Equivalent amounts (0.12 μM) of purified mature wild-type SpeB generated by autocatalysis (filled squares), as well as two-plasmid (filled circles) and polycistronic (filled triangles) co-expression were evaluated by use of a resorufin-labeled casein cleavage assay as described in Example 1. An incubation time of 1 hour at 25° C. was used for cleavage reactions. Purified mature C192S SpeB (open diamonds) produced by polycistronic expression was evaluated as a control.

FIG. 11 demonstrates antibody mediated inhibition of wild-type SpeB proteolytic activity. Increasing amounts of antiserum generated against mature C192S SpeB produced by either the two-plasmid (filled diamonds), or polycistronic system (open squares) was evaluated for the ability to specifically inhibit the proteolytic activity of mature wild-type SpeB using a resorufin-labeled casein cleavage assay. An incubation time of 2 hours at 37° C. was used for cleavage reactions. Pre-immune serum was used as a negative control (tri-star) for analysis.

### DETAILED DESCRIPTION OF THE INVENTION

The invention described hereinafter, addresses a need in the art for methods of producing Streptococcus pyogenes exotoxin B (hereinafter, “SpeB”) for use in immunogenic compositions. In certain preferred embodiments, the invention is directed to methods of producing mature SpeB for use in immunogenic compositions. More particularly, the invention described hereinafter addresses a need in the art for methods of recombinantly expressing mature SpeB polypeptide in a host cell, wherein the expressed mature SpeB polypeptide is soluble in the host cell. In a preferred embodiment, a mature SpeB polypeptide produced according to the methods of the invention is immunogenic when administered to a mammalian host.

As defined hereinafter, a “SpeB zymogen”, as expressed in Streptococcus pyogenes, is a 40 kDa pre-pro-polypeptide comprising amino acids 1-398 of SEQ ID NO:2. The SpeB zymogen is expressed as an enzymatically inactive (ie., a zymogen), 40 kDa, pre-pro-polypeptide with a 27 amino acid NH₂-terminal signal (“pre”) sequence, followed by a 118 amino acid pro-polypeptide (“pro”) sequence, and a 253 amino acid mature polypeptide sequence. Thus, as defined hereinafter, the “pre” sequence of the SpeB zymogen comprises amino acids 1-27 of SEQ ID NO:2. Similarly, as defined hereinafter, a “pro sequence“, a “pro-sequence domain”, a “pro-polypeptide sequence” and a “pro-polypeptide domain” of the SpeB zymogen are used interchangeably, wherein the pro sequence comprises amino acids 28-145 of SEQ ID NO:2.

Upon secretion by the native bacterium (ie., S. pyogenes), the 40 kDa SpeB zymogen undergoes autocatalytic activation resulting in the removal of the 12 kDa NH₂-terminal pro-polypeptide sequence (ie., amino acids 28-145 of SEQ ID NO:2) and formation of the mature, 28 kDa, proteolytically active SpeB polypeptide (or enzyme). As defined hereinafter, a “mature SpeB” polypeptide is a 28 kDa polypeptide comprising amino acids 146-398 of SEQ ID NO:2, wherein the mature SpeB polypeptide has cysteine protease activity. As defined hereinafter, a “mature SpeB” polypeptide and a “mature wild-type SpeB” polypeptide are used interchangeably, both of which refer to the wild-type 28 kDa polypeptide comprising amino acids 146-398 of SEQ ID NO:2, wherein the polypeptide has cysteine protease activity.

The SpeB active site contains a Cys-His pair at amino acid residues 192 and 340 of SEQ ID NO:2. An amino acid substitution (i.e., a mutation) of the single cysteine residue at position 192 to a serine residue (hereinafter, “C192S” or “C192S mutant”) results in a loss of detectable proteolytic activity of the mature C192S SpeB polypeptide. Thus, as defined hereinafter, a “C192S SpeB zymogen” or a “C192S SpeB mutant” comprises amino acids 28-398 of SEQ ID NO:2, wherein the cysteine amino acid residue at position 192 has been mutated to a serine amino acid residue. As defined hereinafter, a “mature C192S SpeB” polypeptide is a 28 kDa polypeptide comprising amino acids 146-398 of SEQ ID NO:2, wherein the cysteine amino acid residue at position 192 has been mutated to a serine amino acid residue, wherein the mature C192S SpeB polypeptide has no cysteine protease activity relative to a mature wild-type SpeB polypeptide.

Thus, in certain embodiments the invention is directed to immunogenic compositions comprising a mature SpeB polypeptide, and more preferably a mature C192S SpeB polypeptide. For example, previous immunological studies with the C192S SpeB mutant have demonstrated that mature C192S SpeB polypeptide (ie., lacking the NH₂-terminal pro-polypeptide sequence) is required for the generation of antibodies with maximum inhibitory activity (i.e., cross-reactivity) towards the mature wild-type SpeB polypeptide (Matsuka et al., 1999). Thus, it is contemplated herein that an effective immunogenic composition for immunizing a mammal against S. pyogenes infection comprises at least a mature C192S SpeB or a mature wild-type SpeB polypeptide antigen. However, it is known in the art that the recombinant expression of mature SpeB polypeptide in an E. coli host cell results exclusively in the production of insoluble SpeB polypeptide aggregates in E. coli (Matsuka et al., 1999).

Thus, in particular embodiments, the present invention is directed to methods that overcome the difficulty of expressing SpeB polypeptides in a host cell which are both “mature” and “soluble”. For example, it is demonstrated in one embodiment of the invention, that the co-expression of a plasmid encoding a mature C192S SpeB polypeptide (or a mature wild-type SpeB) and a plasmid encoding the pro-polypeptide domain (i.e., amino acids 28-145 of SEQ ID NO:2) in a host cell, results in the successful expression of soluble, mature C192S SpeB polypeptide (or mature wild-type SpeB) in the host cell (e.g., see Example 3). Similarly, the recombinant co-expression of a polycistronic plasmid in a host cell, wherein the polycistronic plasmid encodes both the mature C192S SpeB polypeptide (or mature wild-type SpeB) and the pro-polypeptide sequence (amino acids 28-145 of SEQ ID NO:2), also results in the expression of soluble, mature C192S SpeB polypeptide (or mature wild-type SpeB) in the host cell (e.g., see Example 4).

Thus, the invention set forth hereinafter, provides novel methods, and novel compositions thereof, for expressing soluble mature C192S SpeB polypeptides (or mature wild-type SpeB) in a host cell, wherein the soluble mature C192S SpeB polypeptides are particularly useful in immunogenic compositions for immunizing a mammal against S. pyogenes infection. Thus, in certain preferred embodiments, the invention is directed to methods for co-expressing a plasmid encoding a mature C192S SpeB polypeptide (or a mature wild-type SpeB) and a plasmid encoding the pro-polypeptide domain (i.e., amino acids 28-145 of SEQ ID NO:2) in a host cell, wherein the mature SpeB polypeptide is soluble in the host cell. In certain other preferred embodiments, the invention is directed to methods for expressing a polycistronic plasmid in a host cell, wherein the polycistronic plasmid encodes both the mature C192S SpeB polypeptide (or mature wild-type SpeB) and the pro-polypeptide sequence (amino acids 28-145 of SEQ ID NO:2), wherein the mature SpeB polypeptide is soluble in the host cell.

The present invention demonstrates a requirement of the pro-sequence domain to modulate the production of soluble mature SpeB, suggesting that the pro-sequence domain functions as an intramolecular chaperone to direct proper folding of the mature SpeB polypeptide. Association of the pro-sequence domain with either mature wild-type SpeB polypeptide or mature C192S SpeB has a dissociation constant (K_{d}) of approximately 11 nm and 34 nM, respectively (Example 2). These binding values indicate a high affinity between the pro-sequence domain and mature SpeB polypeptide domains. In addition, the molecular chaperone activity of the pro-sequence domain is demonstrated in vitro using urea-denatured mature SpeB (Example 2).

Thus, in particular embodiments, the invention is directed to methods of protein assisted folding of an insoluble mature SpeB aggregate. For example, one embodiment of the invention provides a method for producing a mature SpeB polypeptide comprising the steps of (a) recombinantly expressing in a host cell a plasmid comprising a polynucleotide sequence encoding a mature SpeB polypeptide, wherein the SpeB polypeptide forms an insoluble polypeptide aggregate in the host cell; (b) solubilizing the polypeptide aggregate, wherein the solubilized polypeptide is defined as a non-native mature SpeB polypeptide; (c) refolding the non-native mature SpeB polypeptide in the presence of a chaperone protein, wherein the non-native mature SpeB polypeptide is folded into a native mature SpeB polypeptide; and (d) recovering the native mature SpeB polypeptide.

Similarly, In other embodiments the invention is directed to methods of protein assisted folding, wherein the mature SpeB polypeptide is expressed in the presence of one or more molecular chaperone proteins. For example, the invention provides a method for expressing a mature SpeB polypeptide in a host cell comprising recombinantly expressing in a host cell a polycistronic plasmid comprising (a) a polynucleotide sequence encoding a mature SpeB polypeptide, (b) a polynucleotide sequence encoding a GroEL polypeptide, wherein the mature SpeB polypeptide is soluble in the host cell and (c) recovering the native mature SpeB polypeptide.

A. POLYNUCLEOTIDES ENCODING SPEB ANTIGENS

Isolated and purified Streptococcus pyogenes polynucleotides of the present invention are used in the production of mature SpeB polypeptide antigens. More specifically, in certain embodiments, polynucleotides of the invention encode a mature SpeB polypeptide and a SpeB pro-polypeptide domain, wherein the mature SpeB polypeptide is soluble when expressed in a host cell. Thus, in certain embodiments of the invention, a polynucleotide encodes a mature SpeB polypeptide comprising amino acids 146 through 398 of SEQ ID NO:2 and a second polynucleotide encodes a pro-polypeptide domain comprising amino acids 28 through 145 of SEQ ID NO:2. In a preferred embodiment, a polynucleotide of the invention encodes a mature C912S SpeB polypeptide, wherein the mature C192S SpeB polypeptide comprises amino acids 146 through 398 of SEQ ID NO:2, wherein the cysteine at amino acid residue 192 of SEQ ID NO:2 has been mutated to a serine residue. In certain preferred embodiments, a polynucleotide encoding a mature SpeB polypeptide comprises nucleotides 436 through 1197 of SEQ ID NO:1, a polynucleotide encoding a mature C192S SpeB polypeptide comprises nucleotides 436 through 1197 of SEQ ID NO:1, wherein amino acid residue 192 of SEQ ID NO:2 is a serine amino acid, and a polynucleotide encoding a pro-polypeptide domain comprises nucleotides 82 through 435 of SEQ ID NO:1.

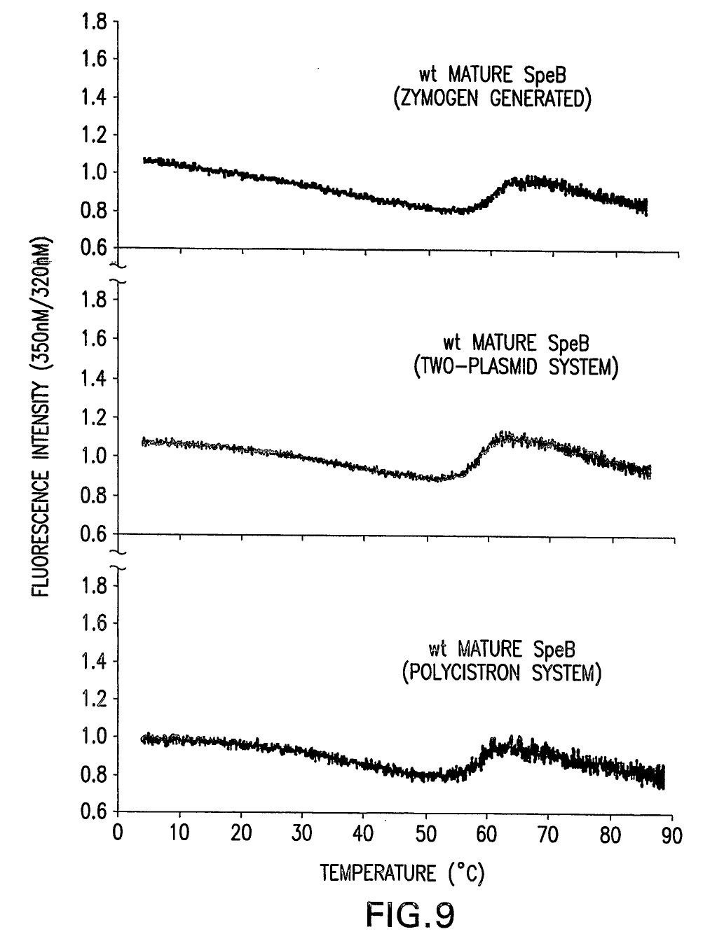

Thus, in particular embodiments, a polynucleotide of the present invention is a DNA molecule, wherein the DNA may be genomic DNA, plasmid DNA or cDNA. In a preferred embodiment, a polynucleotide of the present invention is a recombinant cDNA polynucleotide. In another preferred embodiment, a polynucleotide encoding a mature SpeB polypeptide is comprised in a first plasmid vector and a polynucleotide encoding a SpeB pro-polypeptide domain is comprised in a second plasmid vector, wherein both vectors are co-expressed in a host cell. In another preferred embodiment, a polynucleotide encoding a mature SpeB polypeptide and a polynucleotide encoding a SpeB pro-polypeptide domain are comprised in a polycistronic expression construct. As described in Section E (Example 4 and FIG. 9), a polycistronic construct of the invention comprises, in a 5′ to 3′ direction, a SpeB pro-polypeptide domain as the first cistron, followed by a synthetic linker comprising a translational enhancer and optimized Shine-Dalgarno ribosome binding site and a mature SpeB as the second cistron. In another embodiment, a polycistronic construct of the invention comprises, in a 5′ to 3′ direction, a mature SpeB as the first cistron, followed by a synthetic linker comprising a translational enhancer and optimized Shine-Dalgarno ribosome binding site and a SpeB pro-polypeptide domain as the second cistron.

As used hereinafter, the term “polynucleotide” means a sequence of nucleotides connected by phosphodiester linkages. Polynucleotides are presented hereinafter from the 5′ to the 3′ direction. A polynucleotide of the present invention comprises from about 10 to about several hundred thousand base pairs. Preferably, a polynucleotide comprises from about 10 to about 3,000 base pairs. Preferred lengths of particular polynucleotide are set forth hereinafter.

A polynucleotide of the present invention is a deoxyribonucleic acid (DNA) molecule, a ribonucleic acid (RNA) molecule, or analogs of the DNA or RNA generated using nucleotide analogs. The nucleic acid molecule is single-stranded or double-stranded, but preferably is double-stranded DNA. Where a polynucleotide is a DNA molecule, that molecule is a gene, a cDNA molecule or a genomic DNA molecule. Nucleotide bases are indicated hereinafter by a single letter code: adenine (A), guanine (G), thymine (T), cytosine (C), inosine (I) and uracil (U).

“Isolated” means altered “by the hand of man” from the natural state. An “isolated” composition or substance is one that has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed hereinafter.

Preferably, an “isolated” polynucleotide is free of sequences which naturally flank the nucleic acid (i.e., sequences located at the 5′ and 3′ ends of the nucleic acid) in the genomic DNA of the organism from which the nucleic acid is derived. For example, in various embodiments, an isolated SpeB nucleic acid molecule contains less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. However, a SpeB nucleic acid molecule is fused to other protein encoding or regulatory sequences and still be considered isolated.

SpeB polynucleotides of the present invention are obtained using standard cloning and screening techniques, from a cDNA library derived from mRNA. Polynucleotides of the invention are also obtained from natural sources such as genomic DNA libraries (e.g., a Streptococcus pyogenes library) or are synthesized using well known and commercially available techniques.

Orthologues and allelic variants of polynucleotides encoding mature SpeB and/or the SpeB pro-polypeptide domain are readily identified using methods well known in the art. Allelic variants and orthologues of the polynucleotides comprise a nucleotide sequence that is typically at least about 70-75%, more typically at least about 80-85%, and most typically at least about 90-95% or more homologous to the nucleotide sequence shown in SEQ ID NO:1, or a fragment of this nucleotide sequence. Such nucleic acid molecules are readily identified as being able to hybridize, preferably under stringent conditions, to the nucleotide sequence shown in SEQ ID NO:1, or a fragment of this nucleotide sequence.

Polynucleotides encoding the mature SpeB polypeptide and the SpeB pro-polypeptide domain are used for the recombinant production of soluble mature SpeB polypeptides or fragments thereof in a host cell. The polynucleotides may include the coding sequence for the mature SpeB polypeptide and/or the coding sequence for the SpeB pro-polypeptide domain. The polynucleotide may also contain non-coding 5′ and 3′ sequences, such as transcribed, non-translated sequences, splicing signals, promoter/enhancer sequences, ribosomal binding sites and polyadenylation signals.

Thus, in certain embodiments, the polypeptide sequence information provided by the present invention (i.e., SEQ ID NO:1) allows for the preparation of relatively short DNA (or RNA) oligonucleotide sequences having the ability to specifically hybridize to gene sequences of the selected polynucleotides disclosed hereinafter. The term “oligonucleotide” as used hereinafter is defined as a molecule comprised of two or more deoxyribonucleotides or ribonucleotides, usually more than three (3), and typically more than ten (10) and up to one hundred (100) or more (although preferably between twenty and thirty). The exact size will depend on many factors, which in turn depends on the ultimate function or use of the oligonucleotide. Thus, in particular embodiments of the invention, nucleic acid probes of an appropriate length are prepared based on a consideration of a selected nucleotide sequence. The ability of such nucleic acid probes to specifically hybridize to a polynucleotide encoding a mature SpeB polypeptide or a SpeB pro-polypeptide domain lends them particular utility in a variety of embodiments. Most importantly, the probes are used in a variety of assays for detecting the presence of complementary sequences in a given sample.

In certain embodiments, it is advantageous to use oligonucleotide primers. These primers may be generated in any manner, including chemical synthesis, DNA replication, reverse transcription, or a combination thereof. The sequence of such primers is designed using a polynucleotide of the present invention for use in detecting, amplifying or mutating a defined segment of a SpeB polynucleotide that encodes a SpeB polypeptide from prokaryotic cells using polymerase chain reaction (PCR) technology.

In certain embodiments, it is advantageous to employ a polynucleotide of the present invention in combination with an appropriate label for detecting hybrid formation. A wide variety of appropriate labels are known in the art, including radioactive, enzymatic or other ligands, such as avidin/biotin, which are capable of giving a detectable signal.

To provide certain of the advantages in accordance with the present invention, a preferred nucleic acid sequence employed for hybridization studies or assays includes probe molecules that are complementary to at least a 10 to about 18 nucleotides long stretch of a polynucleotide encoding a polypeptide of SEQ ID NO:2. A size of at least 10 nucleotides in length helps to ensure that the fragment will be of sufficient length to form a duplex molecule that is both stable and selective. Molecules having complementary sequences over stretches greater than 10 bases in length are generally preferred, though, in order to increase stability and selectivity of the hybrid, and thereby improve the quality and degree of specific hybrid molecules obtained. Such fragments are readily prepared by, for example, directly synthesizing the fragment by chemical means, by application of nucleic acid reproduction technology, such as the PCR technology of (U.S. Pat. No. 4,683,202, incorporated hereinafter by reference) or by excising selected DNA fragments from recombinant plasmids containing appropriate inserts and suitable restriction enzyme sites.

Accordingly, a polynucleotide probe molecule of the invention is used for its ability to selectively form duplex molecules with complementary stretches of the gene. Depending on the application envisioned, one will desire to employ varying conditions of hybridization stringency to achieve varying degree of selectivity of the probe toward the target sequence (see Table 1 below). For applications requiring a high degree of selectivity, one will typically desire to employ relatively stringent conditions to form the hybrids. For some applications, for example, where one desires to prepare mutants employing a mutant primer strand hybridized to an underlying template or where one seeks to isolate a homologous polypeptide coding sequence from other cells, functional equivalents, or the like, less stringent hybridization conditions are typically needed to allow formation of the heteroduplex (see Table 1). Cross-hybridizing species are thereby readily identified as positively hybridizing signals with respect to control hybridizations. In any case, it is generally appreciated that conditions are rendered more stringent by the addition of increasing amounts of formamide, which serves to destabilize the hybrid duplex in the same manner as increased temperature. Thus, hybridization conditions are readily manipulated, and thus will generally be a method of choice depending on the desired results.

The present invention also includes polynucleotides capable of hybridizing under reduced stringency conditions, more preferably stringent conditions, and most preferably highly stringent conditions, to polynucleotides described hereinafter. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1
Hybridization Stringency Conditions

| | Poly- | Hybrid | Hybridization | Wash |
|---|---|---|---|---|
| Stringency | nucleotide | Length | Temperature and | Temperature |
| Condition | Hybrid | (bp)^{I} | Buffer^{H} | and Buffer^{H} |
| A | DNA:DNA | >50 | 65° C.; 1 × SSC -or- | 65° C.; |
| | | | 42° C.; 1 × SSC, 50% | 0.3 × SSC |
| | | | formamide | |
| B | DNA:DNA | <50 | T_{B}; 1 × SSC | T_{B}; 1 × SSC |
| C | DNA:RNA | >50 | 67° C.; 1 × SSC -or- | 67° C.; |
| | | | 45° C.; 1 × SSC, 50% | 0.3 × SSC |
| | | | formamide | |
| D | DNA:RNA | <50 | T_{D}; 1 × SSC | T_{D}; 1 × SSC |
| E | RNA:RNA | >50 | 70° C.; 1 × SSC -or- | 70° C.; |
| | | | 50° C.; 1 × SSC, 50% | 0.3 × SSC |
| | | | formamide | |
| F | RNA:RNA | <50 | T_{F}; 1 × SSC | T_{F}; 1 × SSC |
| G | DNA:DNA | >50 | 65° C.; 4 × SSC -or- | 65° C.; |
| | | | 42° C.; 4 × SSC, 50% | 1 × SSC |
| | | | formamide | |
| H | DNA:DNA | <50 | T_{H}; 4 × SSC | T_{H}; 4 × SSC |
| I | DNA:RNA | >50 | 67° C.; 4 × SSC -or- | 67° C.; |
| | | | 45° C.; 4 × SSC, 50% | 1 × SSC |
| | | | formamide | |
| J | DNA:RNA | <50 | T_{J}; 4 × SSC | T_{J}; 4 × SSC |
| K | RNA:RNA | >50 | 70° C.; 4 × SSC -or- | 67° C.; |
| | | | 50° C.; 4 × SSC, 50% | 1 × SSC |
| | | | formamide | |
| L | RNA:RNA | <50 | T_{L}; 2 × SSC | T_{L}; 2 × SSC |
| M | DNA:DNA | >50 | 50° C.; 4 × SSC -or- | 50° C.; |
| | | | 40° C.; 6 × SSC, 50% | 2 × SSC |
| | | | formamide | |
| N | DNA:DNA | <50 | T_{N}; 6 × SSC | T_{N}; 6 × SSC |
| O | DNA:RNA | >50 | 55° C.; 4 × SSC -or- | 55° C.; |
| | | | 42° C.; 6 × SSC, 50% | 2 × SSC |
| | | | formamide | |
| P | DNA:RNA | <50 | T_{P}; 6 × SSC | T_{P}; 6 × SSC |
| Q | RNA:RNA | >50 | 60° C.; 4 × SSC -or- | 60° C.; |
| | | | 45° C.; 6 × SSC, 50% | 2 × SSC |
| | | | formamide | |
| R | RNA:RNA | <50 | T_{R}; 4 × SSC | T_{R}; 4 × SSC |
| (bp)^{I}: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length is determined by aligning the sequences of the polynucleotides and identifying the region or regions of optimal sequence complementarity. | | | | |
| Buffer^{H}: SSPE (1 × SSPE is 0.15 M NaCl, 10 mM NaH₂PO₄, and 1.25 mM EDTA, pH 7.4) is substituted for SSC (1 × SSC is 0.15 M NaCl and 15 mM sodium citrate) in the hybridization and wash buffers; washes are performed for 15 minutes after hybridization is complete. | | | | |
| T_{B} through T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, | | | | |
| # Tₘ(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1 × SSC = 0.165 M). | | | | |

Additional examples of stringency conditions for polynucleotide hybridization are provided in Sambrook et al., 1989, Molecular Cloning: A Laboratory Manual, Cold Spring Harbor Laboratory Press, Cold Spring Harbor, N.Y., chapters 9 and 11, and Ausubel et al., 1995, Current Protocols in Molecular Biology, eds., John Wiley & Sons, Inc., sections 2.10 and 6.3-6.4, incorporated hereinafter by reference.

B. MATURE SPEB POLYPEPTIDE ANTIGENS

In particular embodiments, the present invention provides immunogens comprising mature SpeB polypeptides and/or mature C192S SpeB polypeptides. In certain embodiments, these immunogens are used in immunogenic compositions for immunizing a mammalian host against Streptococcus pyogenes infections. In preferred embodiments, the immunogen is a mature C192S SpeB polypeptide which confers protection (i.e., cross-protection) against a high percentage of heterologous Streptococcus pyogenes strains.

An antigen or immunogen is typically defined on the basis of immunogenicity. Immunogenicity is defined as the ability to induce a humoral and/or cell-mediated immune response. Thus, the terms “antigen” or “immunogen”, as defined hereinafter, are molecules possessing the ability to induce a humoral and/or cell-mediated immune response.

Thus, in one embodiment, the invention is directed to immunogenic compositions comprising at least a Streptococcus pyogenes mature SpeB polypeptide and/or a mature C192S SpeB polypeptide. In preferred embodiments, a mature SpeB polypeptide antigen comprises amino acids 146 through 398 of SEQ ID NO:2. In another preferred embodiment, a mature C192S SpeB polypeptide antigen comprises amino acids 146 through 398 of SEQ ID NO:2, wherein the amino acid at position 192 of SEQ ID NO:2 is mutated from a cysteine to a serine residue. In certain other embodiments of the invention, an immunogenic composition comprising a mature SpeB polypeptide and/or a mature C192S SpeB polypeptide, further comprises one or additional polypeptide antigens (i.e., a polypeptide other than SpeB), wherein the one or more additional polypeptide antigens are Streptococcus pyogenes antigens or antigens from other infectious bacteria and/or viruses.

A biological equivalent or variant of a SpeB polypeptide according to the present invention encompasses a polypeptide that contains substantial homology to a Streptococcus pyogenes polypeptide selected from the group consisting of a SpeB zymogen, a C192S SpeB zymogen, a mature SpeB polypeptide, a mature C192S SpeB polypeptide, and a SpeB pro-polypeptide domain. For example, biological equivalents or variants of the mature SpeB polypeptide and the mature C192S SpeB polypeptide also include those polypeptides where the mature SpeB polypeptide of SEQ ID NO:2 is modified, so long as the mature SpeB polypeptide maintains the ability to elicit an immunogenic response. Generally, functional biological equivalents or variants of the mature SpeB polypeptide are naturally occurring amino acid sequence variants, wherein the mature SpeB polypeptide maintains the ability to elicit an immunogenic response.

Modifications and changes are made in the structure of a mature SpeB polypeptide of the invention and still obtain a molecule having SpeB immunogenic properties. Because it is the interactive capacity and nature of a polypeptide that defines that polypeptide's biological functional activity, certain amino acid sequence substitutions are made in a polypeptide sequence (or, of course, its underlying DNA coding sequence) and nevertheless obtain a polypeptide with like properties.

Amino acid substitutions are generally based on the relative similarity of the amino acid side-chain substituents, for example, their hydrophobicity, hydrophilicity, charge, size, and the like (e.g., see Kyte and Doolittle, 1982 and U.S. Pat. No. 4,554,101, incorporated hereinafter by reference in its entirety). Exemplary substitutions which take various of the foregoing characteristics into consideration are well known to those of skill in the art and include: arginine and lysine; glutamate and aspartate; serine and threonine; glutamine and asparagine; and valine, leucine and isoleucine (See Table 2, below). The present invention thus contemplates functional or biological equivalents of a mature SpeB polypeptide as set forth above.

TABLE 2
AMINO ACID SUBSTITUTIONS

| | Exemplary Residue |
|---|---|
| Original Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

In particular embodiments of the invention, multivalent or combination immunogenic compositions are provided. Combination immunogenic compositions are provided by including one or more of the polypeptides of the invention (e.g., a mature C192S SpeB), or a fragment thereof (e.g., a SpeB epitope fragment), with one or more additional antigens. In particular, combination immunogenic compositions are provided by combining one or more mature SpeB polypeptides, or fragments thereof, with one or more polypeptide, polypeptide fragment, carbohydrate, oligosaccharide, lipid, lipooligosaccharide, polysaccharide, oligosaccharide-protein conjugate, polysaccharide-protein conjugate, peptide-protein conjugate, oligosaccharide-peptide conjugate, polysaccharide-peptide conjugate, protein-protein conjugate, lipooligosaccharide-protein conjugate or polysaccharide-protein conjugate.

Thus, in certain embodiments, one or more antigens set forth above are conjugated to an antigen carrier protein via chemical attachment (i.e., conjugation). Means for conjugating a polypeptide to a carrier protein are well known in the art and include glutaraldehyde, m-maleimidobencoyl-N-hydroxysuccinimide ester, carbodiimide and bis-biazotized benzidine.

Exemplary conventional protein carriers include, without limitation, E. coli DnaK protein, galactokinase (galK), ubiquitin, α-mating factor, β-galactosidase, and influenza NS-1 protein. Toxoids (Le., the sequence which encodes the naturally occurring toxin, with sufficient modifications to eliminate its toxic activity) such as diphtheria toxoid and tetanus toxoid, their respective toxins, and any mutant forms of these proteins, may also be employed as carriers. An exemplary carrier protein is diphtheria toxin CRM₁₉₇ (a non-toxic form of diphtheria toxin, see U.S. Pat. No. 5,614,382, incorporated herein by reference in its entirety). Other carriers include exotoxin A of Pseudomonas, heat labile toxin of E. coli, Vibrio cholera and rotaviral particles (including rotavirus and VP6 particles). Alternatively, a fragment or epitope of the carrier protein or other immunogenic protein may be used. For example, a hapten may be coupled to a T cell epitope of a bacterial toxin (see, U.S. Pat. No. 5,785,973. Similarly a variety of bacterial heat shock proteins, e.g., mycobacterial hsp-70 may be used. Glutathione-S-transferase (GST) is another useful carrier. One of skill in the art can readily select an appropriate carrier for use in this context.

In certain embodiments, the invention is directed to methods of protein assisted folding of an insoluble mature SpeB aggregate. Similarly, in other embodiments the invention is directed to methods of protein assisted folding, wherein the mature SpeB polypeptide is expressed in the presence of one or more molecular chaperone proteins.

For example, in one embodiment the invention provides a method for producing a mature SpeB polypeptide comprising the steps of (a) recombinantly expressing in a host cell a plasmid comprising a polynucleotide sequence encoding a mature SpeB polypeptide, wherein the SpeB polypeptide forms an insoluble polypeptide aggregate in the host cell; (b) solubilizing the polypeptide aggregate, wherein the solubilized polypeptide is defined as a non-native mature SpeB polypeptide; (c) refolding the non-native mature SpeB polypeptide in the presence of a chaperone protein, wherein the non-native mature SpeB polypeptide is folded into a native mature SpeB polypeptide; and (d) recovering the native mature SpeB polypeptide. In certain other embodiments, the invention provides a method for recombinantly expressing a mature SpeB polypeptide in a host cell comprising a polycistronic plasmid comprising (i) a polynucleotide sequence encoding a mature SpeB polypeptide and (ii) a polynucleotide sequence encoding a GroEL polypeptide, wherein the mature SpeB polypeptide is soluble in the host cell.

Thus, in particular embodiments, the invention provides a molecular chaperone protein to assist in the folding of a mature SpeB polypeptide. Molecular chaperones are well known in the art, and include but are not limited to, ribosome binding proteins such as trigger factor (TF); the Hsp7O family of chaperones such as Hsp70, DnaK, Hsp40, DnaJ, GrpE and the Chaperonin family of chaperones such as GroEL, GroES, Hsp60, Hsp10 (Creighton, 1993; Hartl and Hayer-Hartl, 2002). The polynucleotide and polypeptide sequences of the molecular chaperone proteins contemplated for use in the present invention are well known in the art (e.g., see, U.S. Pat. No. 6,159,708; U.S. Pat. No. 6,010,879; U.S. Pat. No. 5,776,724 and Lorimer and Baldwin, Methods in Enzymology, 1998, each incorporated herein by reference in its entirety), as are the folding/refolding requirements, cofactors and the like, for a given molecular chaperone protein as described below.

Thus, by way of a non-limiting example, the molecular chaperonin GroEL from E. coli is a member of the heat shock protein 60 (Hsp60) class of chaperones and is expressed, along with GroES, from the E. coli GroE operon. GroEL assists in protein folding reactions by binding unfolded proteins (e.g., mature SpeB), which decreases the concentration of aggregation-prone polypeptide intermediates and the rate of off-pathway aggregation, thereby favoring partitioning to the native conformation (e.g., properly folded, soluble mature SpeB). It is known that the co-chaperonin GroES and cofactors such as ATP, K⁺ and Mg²⁺ further increase the yield of the GroEL mediated polypeptide folding reaction. Thus, in particular embodiments, the skilled artisan will include components, cofactors, additional chaperone proteins and the like, known in the art to improve or enhance chaperone mediated (or assisted ) protein folding.

Although the mechanism may vary for a given chaperone family or class, the underlying feature shared by all molecular chaperone proteins (with the exception of PDI and PPI set forth below) is an ability to bind a protein in its non-native conformation. As defined hereinafter, a “molecular chaperone” or a “chaperone” protein of the invention is protein which assists the folding of a polypeptide in vivo and/or in vitro.

Also contemplated herein as molecular chaperone “type” proteins are protein disulfide isomerase (PDI) and peptidyl-prolyl cis/trans isomerase (PPI), which catalyze protein disulfide bond formation and proline cis/trans isomerization, respectively (see, Lorimer and Baldwin, Methods in Enzymology, 1998). For example, it was demonstrated by S. pyogenes mutagenesis (Lyon et al., 1998), that the expression of SpeB in S. pyogenes requires the RopA protein. The RopA protein is a molecular chaperone which is known to bind nascent polypeptides on ribosomes, bind GroEL and have peptidyl-prolyl isomerase activity. Thus, in certain other embodiments of the invention, methods for producing a mature SpeB polypeptide comprise refolding a non-native mature SpeB polypeptide or expressing a mature SpeB in the presence of PPI, particularly the RopA PPI.

C. RECOMBINANT EXPRESSION VECTORS AND HOST CELLS

In another embodiment, the present invention is directed to expression vectors comprising polynucleotides that encode a SpeB pro-polypeptide domain and a mature SpeB polypeptide. In certain embodiments, an expression vector of the present invention comprises a polycistronic nucleic acid sequence, wherein one cistron comprises a polynucleotide that encodes a SpeB pro-polypeptide domain and a second cistron comprises a polynucleotide that encodes the mature SpeB polypeptide. In certain other embodiments, an expression vector of the present invention comprises a polynucleotide encoding a SpeB pro-polypeptide domain and a second expression vector comprises a polynucleotide encoding a mature SpeB polypeptide. In one preferred embodiment, an expression vector of the invention is a plasmid construct. In another preferred embodiment, an expression vector of the invention is a plasmid set forth in Table 3, Example 1.

As used herein, the term “vector” refers to a nucleic acid molecule capable of transporting another nucleic acid to which it has been linked. One type of vector is a “plasmid,” which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Expression of proteins in prokaryotes is most often carried out in E. coli with vectors containing constitutive or inducible promoters directing the expression of either fusion or nonfusion proteins. Fusion vectors add a number of amino acids to a protein encoded therein, to the amino or carboxy terminus of the recombinant protein.

In certain embodiments, a host cell is transfected, transformed, transduced or infected with a polycistronic plasmid comprising (i) a polynucleotide sequence encoding a SpeB pro-polypeptide domain and (ii) a polynucleotide sequence encoding a mature SpeB polypeptide. As defined herein, a “polycistronic mRNA” codes for two or more polypeptides. Thus, as defined hereinafter, a “polycistronic polynucleotide”, a polycistronic cDNA” or a “polycistronic plasmid” of the invention codes for a polycistronic mRNA, which in turn encodes two or more polypeptides.

Examples of suitable inducible, non-fusion, E. coli expression vectors include pTrc (Amann et al., 1988), pET Ild (Studier et al., 1990), pET, PRSET, pCRT7-CTTOPO and pIVeX. Target gene expression from the pTrc vector relies on host RNA polymerase transcription from a hybrid trp-lac fusion promoter. Target gene expression from the pET Ild vector relies on transcription from a T7 gn1 β-lac fusion promoter mediated by a coexpressed viral RNA polymerase T7 gnl. This viral polymerase is supplied by host strains BL21 (DE3) or HMS I 74(DE3) from a resident prophage harboring a T7 gnl gene under the transcriptional control of the lacUV 5 promoter. Also contemplated in certain embodiments are plasmid vectors comprising human CMV or simian CMV promoters such as pRK5, pCMVBlue, pCMV-LIC, pAPL 400-023, pAPL 400-087 and pAPL 400-088.

One strategy to maximize recombinant protein expression in E. coli is to express the protein in a host bacteria with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli. Such alteration of nucleic acid sequences of the invention is carried out by standard DNA mutagenesis or synthesis techniques.

A promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region or a promoter region.

Another aspect of the invention pertains to host cells into which a recombinant expression vector of the invention has been introduced. The terms “host cell”, “genetically engineered host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell is any prokaryotic or eukaryotic cell, but is preferably a prokaryotic cell. For example, a SpeB polypeptide is expressed in bacterial cells such as E. coli, and S. pyogenes. In other embodiments, a SpeB polypeptide is expressed in insect cells (e.g., Sf9, high five and Sf21 cells), yeast (e.g., P. pastoris, P. methanolica, S. pombe and S. cerevisiae) or mammalian cells (e.g., Chinese hamster ovary cells (CHO), Cos-1, CV-1, HeLa, NIH3T3, PER-C6 and NSO). Other suitable host cells are known to those skilled in the art.

A host cell of the invention, such as a prokaryotic host cell in culture, is used to produce (i.e., express) SpeB polypeptides. In one embodiment, the method comprises culturing the host cell of invention (into which a recombinant expression vector encoding a mature SpeB polypeptide and a SpeB pro-polypeptide domain has been introduced) in a suitable medium until the mature SpeB polypeptide and SpeB pro-polypeptide domain are produced. In another embodiment, the method further comprises isolating the mature SpeB polypeptide from the medium or the host cell.

Vector DNA is Introduced into prokaryotic or eukaryotic cells via conventional transformation, transduction, infection or transfection techniques. As used herein, the terms “transformation”, “infection”, and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, infection or electroporation. Suitable methods for transforming, infecting or transfecting host cells is found in Sambrook, et al. (“Molecular Cloning: A Laboratory Manual” 2nd ed, Cold Spring Harbor Laboratory, Cold Spring Harbor Laboratory Press, Cold Spring Harbor, N.Y., 1989), and other laboratory manuals.

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells are transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

A coding sequence of an expression vector is operatively linked to a transcription terminating region. RNA polymerase transcribes an encoding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA). Transcription-terminating regions are well known in the art. An exemplary transcription-terminating region comprises a polyadenylation signal of SV40 or the protamine gene.

A DNA molecule, gene or polynucleotide of the present invention are incorporated into a vector by a number of techniques which are well known in the art. For instance, the vector pUC18 has been demonstrated to be of particular value Likewise, the related vectors M13mp18 and M13mp19 are used in certain embodiments of the invention, in particular, in performing dideoxy sequencing.

In a preferred embodiment the recombinant host cells of the present invention are prokaryotic host cells. Preferably, the recombinant host cells of the invention are bacterial cells of the DH5 α strain of Escherichia coli. In general, prokaryotes are preferred for the initial cloning of DNA sequences and constructing the vectors useful in the invention. For example, E. coli K12 strains are particularly useful. Other microbial strains which are used include E. coli B, and E. coliₓ1976 (ATCC No. 31537). These examples are, of course, intended to be illustrative rather than limiting.

Prokaryotes are also used for expression. The aforementioned strains, as well as E. coli strains such as W3110 (ATCC No. 273325), BLR(DE3), BLR(DE3)pLysS, AD494(DE3), AD494(DE3)pLysS, BL21(DE3), BL21(DE3) pLysS, BL21(DE3)pLysE, BL21(DE3)pLacI, BL21trxB(DE3), BL21trxB(DE3)pLysS, HMS174(DE3), HMS174(DE3)pLysS, HMS174(DE3)pLysE, Origami(DE3), Origami(DE3)pLysS, Origami(DE3)pLysE, Origami(DE3)pLacI, OrigamiB(DE3), OrigamiB(DE3)pLysS, OrigamiB(DE3)pLysE, OrigamiB(DE3)pLacI, Rosetta(DE3), Rosetta(DE3)pLysS, Rosetta(DE3)pLysE, Rosetta(DE3)pLacI, Tuner(DE3), Tuner(DE3)pLysS and Tuner(DE3)pLacI, bacilli such as Bacillus subtilis, or other enterobacteriaceae such as Salmonella typhimurium or Serratia marcesans, and various Pseudomonas species are used.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is transformed using pBR322, a plasmid derived from an E. coli species (Bolivar et al. 1977). pBR322 contains genes for ampicillin and tetracycline resistance and thus provides an easy means for identifying transformed cells. The pBR plasmid, or other microbial plasmid or phage must also contain, or be modified to contain, promoters which are used by the microbial organism for expression of its own polypeptides.

Those promoters most commonly used in recombinant DNA construction include the β-lactamase (penicillinase) and lactose promoter systems (Chang et al. 1978; Itakura et al. 1977; Goeddel et al. 1979; Goeddel et al. 1980), the tryptophan (TRP) promoter system (European Application No. EP 0036776; Siebwenlist et al. 1980) and the T7 or T7lac promoter system. While these are the most commonly used, other microbial promoters have been discovered and utilized, and details concerning their nucleotide sequences have been published, enabling a skilled worker to introduce functional promoters into plasmid vectors (Siebwenlist et al. 1980).

Following transfection, the cell is maintained under culture conditions for a period of time sufficient for expression of SpeB polypeptides. Culture conditions are well known in the art and include ionic composition and concentration, temperature, pH and the like. Typically, transfected cells are maintained under culture conditions in a culture medium. Suitable medium for various cell types are well known in the art. In a preferred embodiment, temperature is from about 20° C. to about 50° C., more preferably from about 30° C. to about 40° C. and, even more preferably about 37° C.

The pH is preferably from about a value of 6.0 to a value of about 8.0, more preferably from about a value of about 6.8 to a value of about 7.8 and, most preferably about 7.4. Osmolality is preferably from about 200 milliosmols per liter (mosm/L) to about 400 mosm/l and, more preferably from about 290 mosm/L to about 310 mosm/L. Other biological conditions needed for transfection and expression of an encoded polypeptide are well known in the art.

Transfected cells are maintained for a period of time sufficient for expression of SpeB polypeptides. A suitable time depends inter alia upon the cell type used and is readily determinable by a skilled artisan. Typically, maintenance time is from about 2 to about 14 days.

Recombinant SpeB polypeptides are recovered or collected either from the transfected cells or the medium in which those cells are cultured. Recovery comprises isolating and purifying the speB polypeptides. Isolation and purification techniques for polypeptides are well known in the art and include such procedures as precipitation, filtration, chromatography, electrophoresis and the like.

In yet another embodiment, a SpeB polypeptide of the invention is produced by an in vitro protein translation system, such as a cell-free translation system (e.g., see Betton, 2003; Braun et al., 2002; Jermutus et al., 1998; Kigawa et al., 1999; Kim et al., 1996 and Spirin et al., 1988). For example, U.S. Pat. No. 6,399,323 and U.S. Pat. No 5,478,730, each incorporated herein by reference in its entirety, describe methods, conditions and the like for preparation or production of polypeptides in a cell-free (in vitro) translation system.

D. IMMUNOGENIC COMPOSITIONS

In certain preferred embodiments, the present invention provides mature SpeB immunogenic compositions comprising mature SpeB polypeptide immunogens (i.e., mature wild-type or mature C192S) and physiologically acceptable carriers. More preferably, the immunogenic compositions comprise at least a mature wild-type SpeB polypeptide comprising amino acids 146-398 of SEQ ID NO:2 or a mature C192S SpeB polypeptide comprising amino acids 146-398 of SEQ ID NO:2, wherein the cysteine amino acid 192 of SEQ ID NO:2 has been mutated to a serine. In still other embodiments of the invention, multivalent or combination immunogenic compositions are provided. Combination immunogenic compositions are provided by including one or more of the polypeptides of the invention (e.g., a mature C192S SpeB), with one or more additional antigens from S. pyogenes and/or other bacterial species. In particular, combination immunogenic compositions are provided by combining one or more mature SpeB polypeptides of the invention with one or more polypeptide, polypeptide fragment, carbohydrate, oligosaccharide, lipid, lipooligosaccharide, polysaccharide, oligosaccharide-protein conjugate, polysaccharide-protein conjugate, peptide-protein conjugate, oligosaccharide-peptide conjugate, polysaccharide-peptide conjugate, protein-protein conjugate, lipooligosaccharide-protein conjugate or polysaccharide-protein conjugate.

The mature SpeB polypeptides of the invention are incorporated into immunogenic compositions suitable for administration to a mammalian subject, e.g., a human. Such compositions typically comprise the “immunogenic” composition and a pharmaceutically acceptable carrier. As used hereinafter the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media are used in the compositions of the invention. Supplementary active compounds are also incorporated into the compositions.

An immunogenic composition of the invention is formulated to be compatible with its intended route of administration. Examples of routes of administration include parenteral (e.g., intravenous, intradermal, subcutaneous, intramuscular, intraperitoneal), mucosal (e.g., oral, rectal, intranasal, buccal, vaginal, respiratory) and transdermal (topical). Solutions or suspensions used for parenteral, intradermal, or subcutaneous application include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH is adjusted with acids or bases, such as hydrochloric acid or sodium hydroxide. The parenteral preparation is enclosed in ampoules, disposable syringes or multiple dose vials made of glass or plastic.

Sterile injectable solutions are prepared by incorporating the active compound (e.g., a mature SpeB) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They are enclosed in gelatin capsules or compressed into tablets. For the purpose of oral therapeutic administration, the active compound is incorporated with excipients and used in the form of tablets, troches, or capsules. Oral compositions are also prepared using a fluid carrier for use as a mouthwash, wherein the compound in the fluid carrier is applied orally and swished and expectorated or swallowed. Pharmaceutically compatible binding agents, and/or adjuvant materials are included as part of the composition. The tablets, pills, capsules, troches and the like contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Systemic administration is also by mucosal or transdermal means. For mucosal or transdermal administration, penetrants appropriate to the barrier to be permeated are used in the formulation. Such penetrants are generally known in the art, and include, for example, for mucosal administration, detergents, bile salts, and fusidic acid derivatives. Mucosal administration is accomplished through the use of nasal sprays or suppositories. For transdermal administration, the active compounds are formulated into ointments, salves, gels, or creams as generally known in the art.

The compounds are also prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

Biodegradable, biocompatible polymers are used, such as ethylene vinyl acetate, polyanhydrides, polyglycolic acid, collagen, polyorthoesters, and polylactic acid. Methods for preparation of such formulations will be apparent to those skilled in the art. The materials are also obtained commercially from Alza Corporation and Nova Pharmaceuticals, Inc. Liposomal suspensions (including liposomes targeted to infected cells with monoclonal antibodies to viral antigens) are also used as pharmaceutically acceptable carriers. These are prepared according to methods known to those skilled in the art, for example, as described in U.S. Pat. No. 4,522,811 which is incorporated hereinafter by reference.

It is especially advantageous to formulate oral or parenteral compositions in dosage unit form for ease of administration and uniformity of dosage. Dosage unit form as used hereinafter refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

A pharmaceutically acceptable vehicle is understood to designate a compound or a combination of compounds entering into a pharmaceutical or immunogenic composition which does not cause side effects and which makes it possible, for example, to facilitate the administration of the active compound, to increase its life and/or its efficacy in the body, to increase its solubility in solution or alternatively to enhance its preservation. These pharmaceutically acceptable vehicles are well known and will be adapted by persons skilled in the art according to the nature and the mode of administration of the active compound chosen.

The immunogenic compositions of the invention may further comprise one or more adjuvants. An “adjuvant” is a substance that serves to enhance the immunogenicity of an antigen. Thus, adjuvants are often given to boost the immune response and are well known to the skilled artisan. Examples of adjuvants contemplated in the present invention include, but are not limited to, aluminum salts (alum) such as aluminum phosphate and aluminum hydroxide, Mycobacterium tuberculosis, Bordetella pertussis, bacterial lipopolysaccharides, aminoalkyl glucosamine phosphate compounds (AGP), or derivatives or analogs thereof, which are available from Corixa (Hamilton, Mont.), and which are described in U.S. Pat. No. 6,113,918; one such AGP is 2-[(R)-3-Tetradecanoyloxytetradecanoylamino]ethyl 2-Deoxy-4-O-phosphono-3-O-[(R)-3-tetradecanoyoxytetradecanoyl]-2-[(R)-3-tetradecanoyoxytetradecanoylamino]-b-D-glucopyranoside, which is also known as 529 (formerly known as RC529), which is formulated as an aqueous form or as a stable emulsion, MPL™ (3-O-deacylated monophosphoryl lipid A) (Corixa) described in U.S. Pat. No. 4,912,094, synthetic polynucleotides such as oligonucleotides containing a CpG motif (U.S. Pat. No. 6,207,646), polypeptides, saponins such as Quil A or STIMULON™ QS-21 (Antigenics, Framingham, Massachusetts), described in U.S. Pat. No. 5,057,540, a pertussis toxin (PT), or an E. coli heat-labile toxin (LT), particularly LT-K63, LT-R72, CT-S109, PT-K9/G129; see, e.g., International Patent Publication Nos. WO 93/13302 and WO 92/19265, cholera toxin (either in a wild-type or mutant form, e.g., wherein the glutamic acid at amino acid position 29 is replaced by another amino acid, preferably a histidine, in accordance with published International Patent Application number WO 00/18434). Various cytokines and lymphokines are suitable for use as adjuvants. One such adjuvant is granulocyte-macrophage colony stimulating factor (GM-CSF), which has a nucleotide sequence as described in U.S. Pat. No. 5,078,996. A plasmid containing GM-CSF cDNA has been transformed into E. coli and has been deposited with the American Type Culture Collection (ATCC), 1081 University Boulevard, Manassas, Va. 20110-2209, under Accession Number 39900. The cytokine Interleukin-12 (IL-12) is another adjuvant which is described in U.S. Pat. No. 5,723,127. Other cytokines or lymphokines have been shown to have immune modulating activity, including, but not limited to, the interleukins 1-α, 1-β, 2, 4, 5,6, 7, 8, 10, 13, 14,15, 16, 17 and 18, the interferons-α, β and γ, granulocyte colony stimulating factor, and the tumor necrosis factors α and β, and are suitable for use as adjuvants.

A composition of the present invention is typically administered parenterally in dosage unit formulations containing standard, well-known nontoxic physiologically acceptable carriers, adjuvants, and vehicles as desired. The term parenteral as used hereinafter includes intravenous, subcutaneous, intradermal, intramuscular, intraarterial injection, or infusion techniques.

Injectable preparations, for example sterile injectable aqueous or oleaginous suspensions, are formulated according to the known art using suitable dispersing or wetting agents and suspending agents. The sterile injectable preparation are also a sterile injectable solution or suspension in a nontoxic parenterally acceptable diluent or solvent, for example, as a solution in 1,3-butanediol.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil is employed including synthetic mono- or di-glycerides. In addition, fatty acids such as oleic acid find use in the preparation of injectables.

Preferred carriers include neutral saline solutions buffered with phosphate, lactate, Tris, and the like. When administering viral vectors, the vector is purified sufficiently to render it essentially free of undesirable contaminants, such as defective interfering adenovirus particles or endotoxins and other pyrogens, so that it does not cause any untoward reactions in the individual receiving the vector construct. A preferred means of purifying the vector involves the use of buoyant density gradients, such as cesium chloride gradient centrifugation.

A carrier is also a liposome. Means for using liposomes as delivery vehicles are well known in the art.

In particular embodiments, an immunogenic composition of this invention comprises a polynucleotide sequence of this invention operatively associated with a regulatory sequence that controls gene expression. The polynucleotide sequence of interest is engineered into an expression vector, such as a plasmid, under the control of regulatory elements which will promote expression of the DNA, that is, promoter and/or enhancer elements. In a preferred embodiment, the human cytomegalovirus immediate-early promoter/enhancer is used (U.S. Pat. No. 5,168,062). The promoter may be cell-specific and permit substantial transcription of the polynucleotide only in predetermined cells.

The polynucleotide is introduced directly into the host either as “naked” DNA (U.S. Pat. No. 5,580,859) or formulated in compositions with agents which facilitate immunization, such as bupivicaine and other local anesthetics (U.S. Pat. No. 5,593,972) and cationic polyamines (U.S. Pat. No. 6,127,170).

In this polynucleotide immunization procedure, the polypeptides of the invention are expressed on a transient basis in vivo; no genetic material is inserted or integrated into the chromosomes of the host. This procedure is to be distinguished from gene therapy, where the goal is to insert or integrate the genetic material of interest into the chromosome. An assay is used to confirm that the polynucleotides administered by immunization do not give rise to a transformed phenotype in the host (U.S. Pat. No. 6,168,918).

All patents and publications cited herein are hereby incorporated by reference.

### E. EXAMPLES

The following examples are carried out using standard techniques, which are well known and routine to those of skill in the art, except where otherwise described in detail. The following examples are presented for illustrative purpose, and should not be construed in any way as limiting the scope of this invention.

### Example 1

### Materials and Methods

Media and reagents. E. coli BLR(DE3) (Novagen, Calif.) was used for all expression studies. Bacteria were grown in Luria broth (LB) under appropriate antibiotic selection conditions. Ampicillin was used at a concentration of 100 μg/mL and kanamycin at 50 μg/mL. ZeroBluntTOPO cloning vector pCR-Blunt (InVitrogen, Carlsbad, Calif.) was used for cloning of PCR generated fragments. Century-Plus RNA Markers™, Millennium RNA Markers™, RNAlater™, RNAqueous-Midi™, DNA-free™, ULTRAhyb™, NorthernMax™ and RETROscript™ were obtained from Ambion (Austin, Tex.). GeneScreen™ hybridization membrane, Flurorescein-N⁶-dATP, Renaissance® Antifluorescein-AP conjugated polyclonal antibody, and CDP-Star® were acquired from PerkinElmer Life Sciences, Boston, Mass. All restriction enzymes were procured from New England Biolabs (Beverly, Mass.).

Polymerase Chain Reaction. Unless noted otherwise, PCR amplifications were performed in a 50 μL final volume utilizing the following reaction conditions: 0.2 mM dNTPs, 1.0 mM DTT, 0.8 μM each primer, 10 U thermostable polymerase, and 1× thermostable polymerase buffer. For cloning and mutagenesis reactions, Pwo polymerase (Boehringer Mannheim, Indianapolis, Ind.) was employed, while all other amplifications used Taq polymerase (Applied Biosystems, Foster City, Calif.). Amplifications consisted of twenty-five cycles of 94° C. for thirty seconds, 55° C. for thirty seconds and 72° C. for thirty seconds.

Plasmid constructs. A 770 bp fragment corresponding to the mature SpeB coding region was PCR amplified using forward (5′ CCATGGAACCAGTTGTTAAATCTCTCC 3′) (SEQ ID NO:3) and reverse (5′ GGATCCTAAGGTTTGATGCCTACAACAGC 3′) (SEQ ID NO:4) primers containing Nco I and BamH I sites (underlined), respectively. The forward primer was designed to contain an ATG translational start codon nested within the Nco I cloning site, resulting in an added methionine residue to the N-terminus of the expressed protein and altering the first residue from glutamine (CM) to glutamate (GM). Similarly, a 367 bp fragment encompassing the pro-sequence domain (amino acids 28-146) was amplified using forward (5′ CCATGGATCAAAACTTTGCTCGTMCG 3′) (SEQ ID NO:5) and reverse (5′ GGATCCTTATTTAATCTCAGCGGTACCAGC 3′) (SEQ ID NO:6) primers with engineered Nco I (forward) and BamH I (reverse) sites for cloning purposes. A stop codon was inserted immediately 3′ to the BamH I site in the reverse primer to direct translational termination of the expressed recombinant protein. PCR reactions used a plasmid-based template containing the SpeB zymogen with a TGT to AGT mutation encoding a single cysteine to serine substitution at position 192 (C192S), as described previously (Matsuka et al, 1999). PCR products were subcloned into pCR-Blunt and subsequently excised by restriction digestion using Nco I and BamH I. The 770 bp mature C192S SpeB and 367 bp pro-sequence domain coding fragments were purified by agarose gel electrophoresis and ligated into pET28a and pET3d, respectively, using Nco I and BamH I restriction sites. The resulting expression plasmids, pLP681 and pLP682, were co-transformed into E. coli BLR(DE3) using standard methods. The produced bacterial expression strain was utilized for two-plasmid based co-expression analyses.

The mature wild-type SpeB expression construct was generated through the use of mutational PCR employing overlapping forward (5′ GCTACAGGATGTGTTGCTACTGC 3′) (SEQ ID NO:7) and reverse (5′ GCAGTAGCMCACATCCTGTAGC 3′) (SEQ ID NO:8) primers with a single A to T base change (bold) to revert the C192S mutation in pLP681 which was used as a template for PCR. Mutagenesis consisted of a modification of the method described by Weiner et. al. (1994), using the following cycling conditions: one minute 94° C., sixteen cycles of fifteen seconds 94° C. and ten minutes 68° C., followed by twelve cycles of fifteen seconds 94° C. and ten minutes 68° C. with extension time at 68° C. increasing in fifteen second increments upon each cycle, resulting in a final extension time of thirteen minutes at 68° C. The PCR reaction was cut with Dpn I before transformation into E. coli. The resulting clone pLP680 was sequenced prior to use to verify the desired mutation. The plasmid was co-transformed into E. coli BLR(DE3) with pLP682 for use with the two-plasmid based co-expression studies.

To construct the polycistronic expression vectors, the SpeB pro-sequence domain coding region from pLP682 was excised by restriction digest using Nco I and BamH I. The 367 bp fragment was purified by agarose gel electrophoresis and ligated into pET28a also restricted with Nco I and BamH I, resulting in pLP688.

Four different linker regions of increasing length, followed by the mature SpeB coding region were generated by PCR using the following forward primers: 5′ AGATCTAAGGAGATATACATATGGACCCAG 3′ (5 nt linker; SEQ ID NO:9); 5′ AGATCTTTAAGAAGGAGATATACATATGGMCC 3′ (10 nt linker; SEQ ID NO:10); 5′ AGATCTGCACATAACTTTAAGAAGGAGATATACATATGG 3′ (20 nt linker; SEQ ID NO:11); 5′ AGATCTAACTTGACTAAATTCGAACAGCACATAACTTTAAGAAGG AGATATACATATGG 3′ (40 nt linker; SEQ ID NO:12). All forward primers contained a Bgl II restriction site on the 5′ terminus (underlined), an optimized Shine Dalgarno site (bold italic) and translational start codon (bold). A reverse primer (5′CTCGAGCTAAGGTTTGATGCCTA-CAACAGC 3′) (SEQ ID NO:13) containing an Xho I site (underlined) immediately 5′ to the translational stop codon, was employed for all linker-based amplification reactions.

The primers indicated above were used to amplify the 5 nt, 10 nt, or 20 nt linker plus mature C192S SpeB coding region, utilizing pLP681 as a template for PCR. Similarly, pLP680 was used as a template to generate the 20 nt linker plus mature wild-type SpeB PCR product. PCR fragments were subcloned into pCR-Blunt, excised by restriction digest with Bgl II and Xho 1, and purified by agarose gel electrophoresis. Following isolation, fragments were ligated to pLP688, using BamH 1 and Xho I restriction sites, to generate pLP683, pLP684, pLP685, and the pLP687 polycistronic expression constructs. In an identical manner, pLP686 was synthesized using pLP685 as a template for PCR with the primers indicated above. Specifications for all expression plasmids generated and used for in vivo analyses are shown in Table 3. All expression constructs listed in Table 3 are in a pET28a background unless otherwise noted.

TABLE 3
SPECIFICATIONS OF CYSTEINE PROTEASE AND
PRO-SEQUENCE DOMAIN EXPRESSION CONSTRUCTS

| Construct | Expressed Protein(s) |
|---|---|
| pLP680 | Mature wt Cysteine protease |
| pLP681 | Mature C192S Cysteine protease |
| pLP682 | Cysteine protease pro-sequence domain in a pET3d plasmid |
| | background |
| pLP683 | 5 nt linker-containing C192S polycistron; co-expresses both |
| | the pro-sequence domain and mature C192S Cysteine protease |
| pLP684 | 10 nt linker-containing C192S polycistron; co-expresses both |
| | the pro-sequence domain and mature C192S Cysteine protease |
| pLP685 | 20 nt linker-containing C192S polycistron, co-expresses both |
| | the pro-sequence domain and mature C192S Cysteine protease |
| pLP686 | 40 nt linker-containing C192S polycistron; co-expresses both |
| | the pro-sequence domain and mature C192S Cysteine protease |
| pLP687 | 20 nt linker-containing wt polycistron; co-expresses both the |
| | pro-sequence domain and mature wt Cysteine protease |
| pLP688 | Cysteine protease pro-sequence domain |

Expression of recombinant proteins. For both polycistronic and two-plasmid based co-expression studies, 200 mL of LB containing the appropriate antibiotic were inoculated with the desired bacterial expression stock, and grown at 37° C. overnight. The overnight cultures were diluted 1:10 into 2 L of fresh antibiotic containing media, grown at 25° C. to an OD₆₀₀ of approximately 0.6, and induced with 1 mM IPTG for sixteen hours at 25° C. Cells were collected by centrifugation and the resulting cell pellets were stored at −20° C. until use. Both pre- and post-induction samples were taken for analysis of protein expression and RNA isolation. Samples for RNA analysis were stored in RNAlater™ at −70° C. until use.

Protein expression was evaluated by SDS-PAGE analysis following cell lysis by sonication. Insoluble material was pelleted by centrifugation and soluble supernatant fractions were recovered. The cell debris was washed twice with PBS before resuspension in 1 mL of the same. All soluble and insoluble fractions were standardized on the basis of OD₆₀₀ of the bacterial culture before cell lysis. Protein expression was visualized by Coomassie blue staining and by Western blot analysis following transfer to PVDF membrane by standard methods. Blots were probed using a polyclonal antibody generated against the zymogen form of the SpeB, allowing simultaneous detection of both expressed pro-sequence domain and mature 28 kDa SpeB.

Isolation of RNA. Total RNA was isolated from bacterial cultures using RNAqueous-Midi™. Post-induction samples stored in RNAlater™ were thawed at room temperature, and cells were pelleted prior to resuspension in 1 mL RNAqueous™ Lysis/Binding buffer. All lysates were then treated as specified in the instruction manual. Isolated RNA was precipitated using LiCl and treated twice with DNA-free™, as per the manufacturer's specifications, to remove any contaminating genomic or plasmid DNA. RNA was quantitated by analysis of absorbance at 260 nm, and purity assessed by determination of the absorbance ratio A_{260 nm}/A_{280 nm}. RNA samples (1 μg) were analyzed by PCR, using the appropriate primer sets described previously, to verify the absence of contaminating plasmid DNA. In addition, all samples were assessed for residual DNase contamination by spike recovery analysis of 1 ng pLP685 plasmid added to PCR reactions. Only samples proven free of DNA and DNase contamination were used for further analysis.

Northern Blot hybridization. Total RNA (5 μg) was fractionated on a 1% agarose gel under denaturing conditions, using the NorthernMax™ system as specified by the manufacturer. Millennium and Century RNA markers (2 μg each) were pre-stained with EtBr and used to assess RNA size. Samples were transferred to a nylon membrane (GeneScreen™), UV cross-linked, and baked at 80° C. for two hours to reverse the formaldehyde reaction used to fractionate the RNA. RNA standards were visualized by UV, and the fragment positions were indicated on the nylon membrane prior to hybridization. The membrane was pre-wet with 2× SSC (0.3 M NaCl, 0.03 M sodium citrate) and pre-hybridized in ULTRAhyb™ at 42° C. for 2 hours. Probes for Northern analysis were PCR synthesized using pLP685 as a template, and primer sets specific for the pro-sequence domain (367 bp) and mature SpeB sequence (770 bp) as detailed above. Flurorescein-N⁶-dATP (10 μM) was used in PCR reactions to produce randomly labeled mature SpeB and pro-sequence domain DNA probes. The resultant probes were purified by agarose gel electrophoresis, and 32 pg of each denatured, and used for membrane hybridization at 42° C. overnight in ULTRAhyb™. Post-hybridization, the membrane was washed twice with excess 2× SSC for ten minutes at room temperature, twice with 2× SSC, 1% SDS for twenty minutes at 42° C., and twice with 0.2× SSC, 0.1% SDS for twenty minutes at 42° C. The membrane was blocked in BLOTTO and developed using an alkaline phosphatase-conjugated anti-fluorescein polyclonal antibody and CDP-Star®, as per the manufacturer's specifications. The blot was exposed to BioMax MR-2 autoradiographic film (Eastman Kodak, Rochester, N.Y.) for five minutes at room temperature to allow for signal detection.

Synthesis of cDNA. cDNA was prepared from 2 μg of total RNA by reverse transcription using a RETROscript™ First Strand Synthesis kit for RT-PCR and random decamers under conditions specified by the manufacturer. Negative control samples were produced using identical conditions, but in the absence of reverse transcriptase (-RT). Reaction products were diluted 1:200 in nuclease free water prior to use for PCR and quantitative PCR analysis.

Quantitative PCR (qPCR) analysis of cDNA. Primers and probes specific for the pro-sequence domain, the mature SpeB, and the pET28a encoded kanamycin resistance gene (KanR), were designed using PrimerExpress software (Applied Biosystems, Foster City, Calif.). Quantitative PCR reactions were performed under the following conditions: 300 nM each primer (forward and reverse), 200 nM FAM/TAMRA probe, 2× TaqMan Universal PCR Master Mix (Applied Biosystems), and 1 μL diluted cDNA or negative control sample. Reactions were carried out in a 25 μL final volume using cycling conditions of: 50° C. for two minutes, 95° C. for ten minutes, forty cycles of 95° C. for fifteen seconds and 60° C. for one minute on an ABI 7000 Sequence Detection System. KanR cDNA levels were used as an internal control and all reactions standardized on the basis of KanR threshold cycle (Ct). Results are expressed as normalized Ct values.

PCR analysis of cDNA. Diluted cDNA and negative control samples were analyzed by PCR to further assess mRNA transcripts produced from expression plasmids. The PCR primers described above specific for the pro-sequence domain, mature SpeB, and a third set consisting of the pro-sequence domain forward (5′ CCATGGATCAAAACTTTGCTCGTAACG 3′) (SEQ ID NO:14) and mature protease reverse (5′ CTCGAGCTAAGGTTTGATGCCTACAACAGC 3′) (SEQ ID NO:15) primers were used to amplify the pro-sequence domain, mature SpeB, and full-length SpeB zymogen (1119 bp) and polycistronic cDNA (1145-1180 bp), respectively. One μL of each diluted sample was used under conditions described above, with the exception that the amplification cycles were increased to thirty, and the PCR products were analyzed by agarose gel electrophoresis.

Purification of the SpeB pro-sequence domain. Cells were resuspended in lysis buffer (20 mM Tris, pH 7.2, 10 mM MgCl₂, 10 μg/μL DNase) at a ratio of 15 mL buffer/g cells and lysed using a M110-Y Microfluidizer (Microfluidics, Newton, Mass.). Cell debris was pelleted by centrifugation, and the soluble fraction recovered and dialyzed against 50 mM glycine-HCl (pH 3.2) at 4° C. overnight. Dialysis was accompanied by a significant precipitation of E coli proteins. Precipitated material was removed by centrifugation, and the recovered supernatant shifted to pH 4.5 before dialysis against 100 mM sodium acetate (pH 4.5). The sample was loaded onto a SP-Sepharose cation exchange column, and recombinant pro-sequence domain protein eluted with a gradient of 100 mM sodium acetate (pH 4.5), 1 M sodium chloride. Fractions containing the pro-sequence domain were pooled, purity checked by SDS-PAGE electrophoresis, and the protein concentration determined by BCA assay (Smith et al., 1985).

Purification of recombinantly expressed mature wild-type SpeB. Cells were resuspended in lysis buffer (20 mM Tris, pH 7.2, 10 mM MgCl₂, 10 μg/μL DNase) at a ratio of 15 mL buffer/g cells and lysed by microfluidization. Cell debris was removed by centrifugation, and the insoluble mature wild-type SpeB-containing fraction was recovered. The pellet was treated to three consecutive washes (50 mM Tris, pH 8.0, 150 mM NaCl, 1 mM EDTA, 2% Triton X-100) at 4° C. overnight, 1.5 hours at room temperature, and 3.5 hours at room temperature, all with gentle agitation. The pellet was solubilized in 20 mM Tris, pH 8.0, 8 M urea at room temperature overnight with agitation, and the pH shifted to 4.5 with sodium acetate prior to sample application on a SP-Sepharose column equilibrated with 100 mM sodium acetate, pH 4.5, 8 M urea. Recombinant mature wild-type SpeB protein was eluted with a 0 to 750 mM gradient of sodium chloride, purity analyzed by SDS-PAGE electrophoresis, and protein concentration determined by BCA. The purified, denatured protein was utilized for in vitro refolding experiments.

Purification of co-expressed mature SpeB. The purification procedure for both recombinant mature wild-type SpeB and mature C192S SpeB, co-expressed by either the polycistronic or two-plasmid system, was identical. Induced bacterial cell pellets were resuspended in lysis buffer (20 mM Tris, pH 7.2, 10 mM MgCl₂, 10 μg/μL DNase) at a ratio of 15 mL buffer/g cells and lysed by microfluidization. The cell debris was pelleted by centrifugation, and the soluble fraction recovered and dialyzed against 50 mM glycine-HCl (pH 3.2) at 4° C. overnight. Dialysis was accompanied by significant precipitation of E. coli proteins, and was clarified by centrifugation before quickly shifting the pH to 4.5. The sample was diluted 1:1 with 10 M urea and loaded onto a SP-Sepharose column equilibrated with 100 mM sodium acetate (pH 4.5), 5 M urea. Unbound material was washed from the column with equilibration buffer until A_{280 nm} reached baseline. The column was washed with 100 mM sodium acetate (pH 4.5) to remove the urea, and the recombinant mature protease eluted with a gradient of 0 to 1.0 M sodium chloride. The recovered protein was dialyzed against PBS (pH 7.4), purity assessed by SDS-PAGE gel electrophoresis, and protein concentration determined by BCA.

Generation of polyclonal antisera. Purified mature C192S SpeB (produced via polycistronic or two-plasmid based co-expression) was used for generation of antisera. Swiss Webster mice were immunized at weeks 0, 4, and 6 with 5 μg of purified protein using 50 mg MPL and 100 μg AlPO₄ as adjuvants. Animals were bled at week 7.

Interaction of mature SpeB with the pro-sequence domain. The dissociation constant (K_{d}) for the SpeB pro-sequence domain/mature SpeB interaction was determined using an Enzyme-Linked ImmunoSorbent binding Assay (ELISA) or a Biocore 3000 apparatus. For ELISA analysis, increasing concentrations of pepsin-generated recombinant mature C192S SpeB (Matsuka et al., 1999) and recombinant mature wild-type SpeB, inhibited with 20 μM E-64, were incubated in microtiter wells coated with either purified pro-sequence domain, or lysozyme (negative control). Plates were washed with TBS, pH 7.4, 0.05% Tween 20, and bound SpeB detected with affinity purified polyclonal antibody generated against the mature SpeB by measuring absorbance at 405 nm. The resulting concentration-dependant increase in absorbance (A) was fitted to the equation ΔA=Aₘₐₓ+[L]/K_{d}+[L] where K_{d} is the dissociation constant and [L] the concentration of free ligand.

Real-time interaction of the pro-sequence domain with the pepsin-generated recombinant mature C192S SpeB was demonstrated by surface plasmon resonance (SPR) using a Biocore 3000 (Biocore, Piscataway, N.J.). Purified pro-sequence domain was covalently coupled to the activated carboxymethyl dextran-coated biosensor chip according to the manufacturer's specifications. Binding experiments were performed in TBS (pH 7.4), 0.05% Tween 20 at 25° C. Increasing concentrations of pepsin-generated recombinant mature C192S SpeB were added to the immobilized pro-sequence domain, and their association monitored in real time. Sensograms of the association process were analyzed using software supplied with the instrument.

Evaluation of the inhibitory activity of the SpeB pro-sequence domain. Mature SpeB (0.1 μM) was incubated for 1 hour at 25° C. in PBS (pH 7.4), 10 mM DTT in the presence of a resorufin-labeled casein substrate (0.4%) utilizing increasing concentrations of the pro-sequence domain, or lysozyme (negative control). After incubation, undigested substrate was removed by precipitation using 2% trichloroacetic acid and the absorbance of released resorufin-labeled peptides in clarified supernatant fractions measured spectrophotometrically at 574 nm. In addition, a closely related cysteine protease (papain) was analyzed under identical conditions as a negative control to demonstrate the specificity of the pro-sequence domain/mature SpeB interaction.

In vitro refolding of denatured mature SpeB. Recombinant denatured mature SpeB in 100 mM sodium acetate (pH 4.5), 8 M urea, was rapidly diluted (1:20 v/v) with either PBS (pH 7.4), or PBS (pH 7.4) containing 0.5 M arginine. Dilutions were performed with, or without, 20 μM E-64 inhibitor in the presence of increasing concentrations of purified pro-sequence domain as indicated. After dilution, 10 mM DTT was added to each reaction and samples incubated at 4° C. for twenty-four hours. The final concentration of the SpeB in reactions was 5 μM. Following incubation, a 100 μL reaction aliquot was assessed through the use of the resorufin-labeled casein cleavage assay to evaluate activity of the refolded SpeB.

Caseinolytic activity of mature SpeB. To assess the proteolytic activity of the enzyme, denoted amounts of SpeB were incubated in the presence of 0.4% resorufin-labeled casein PBS (pH 7.4), 10 mM DTT, as specified. Undigested substrate was removed by trichloroacetic acid precipitation (2%), samples clarified by centrifugation, and the absorbance of released resorufin-labeled peptides in supernatant fractions determined spectrophotometrically at 574 nm.

Heat-induced denaturation of mature SpeB. Melting of recombinantly expressed mature SpeB was evaluated by heating protein samples in TBS (pH 7.4) while monitoring the ratio of intrinsic fluorescence Intensity at 350 nm/320 nm, with excitation at 280 nm, using a SLM AB2 spectrofluorometer.

### Example 2

### Inhibitory and Chaperone Activities of the SpeB Pro-Sequence Domain

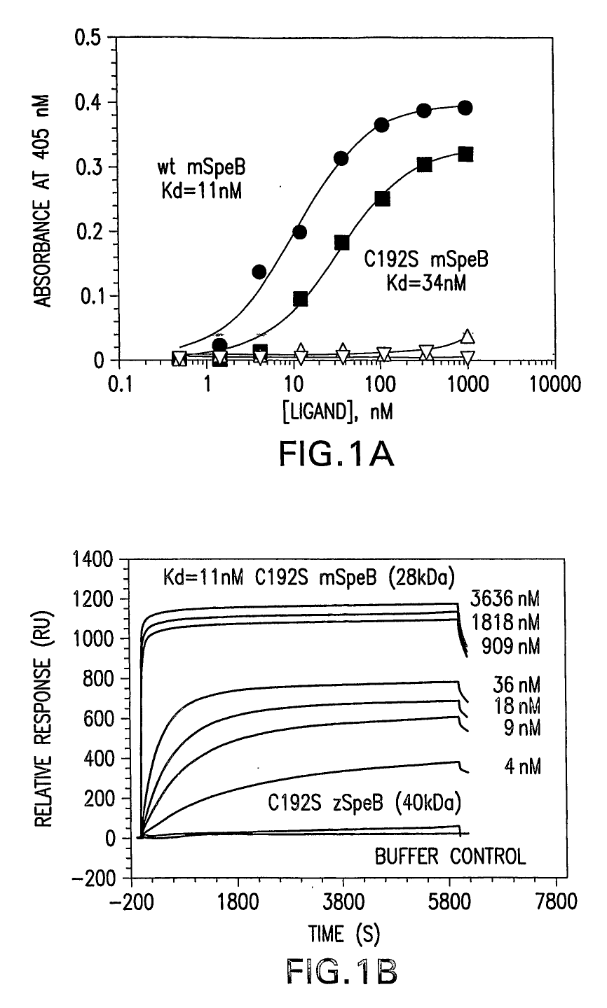

Recombinant expression of mature SpeB, lacking its NH₂-terminal pro-sequence domain, results exclusively in the production of insoluble protein in E. coli (Matsuka et al., 1999). The requirement of the pro-sequence domain to govern production of soluble mature SpeB suggests that the domain might function as an intramolecular chaperone to direct proper folding of the protein. To examine such activity, the SpeB pro-sequence domain, the 40 kDa SpeB zymogen, as well as mature wild-type SpeB and mature C192S SpeB, were expressed and purified from E. coli for characterization (data not shown). Association of the pro-sequence domain and mature SpeB proteins was investigated through the use of ELISA (FIG. 1A) and surface plasmon resonance (SPR) using a Biocore 3000 (FIG. 1B). Using the calculation parameters specified in Example 1, the interaction of the pro-sequence domain with the wild-type and C192S mature SpeB was estimated to have a K_{d} of 11 nm and 34 nM, respectively. Real-time analysis of interactions of the pro-sequence domain and mature C192S SpeB, as determined by SPR, was estimated to have a K_{d} of 11 nM. The binding values determined by both methods indicate a high affinity between the pro-sequence domain and mature SpeB domains.

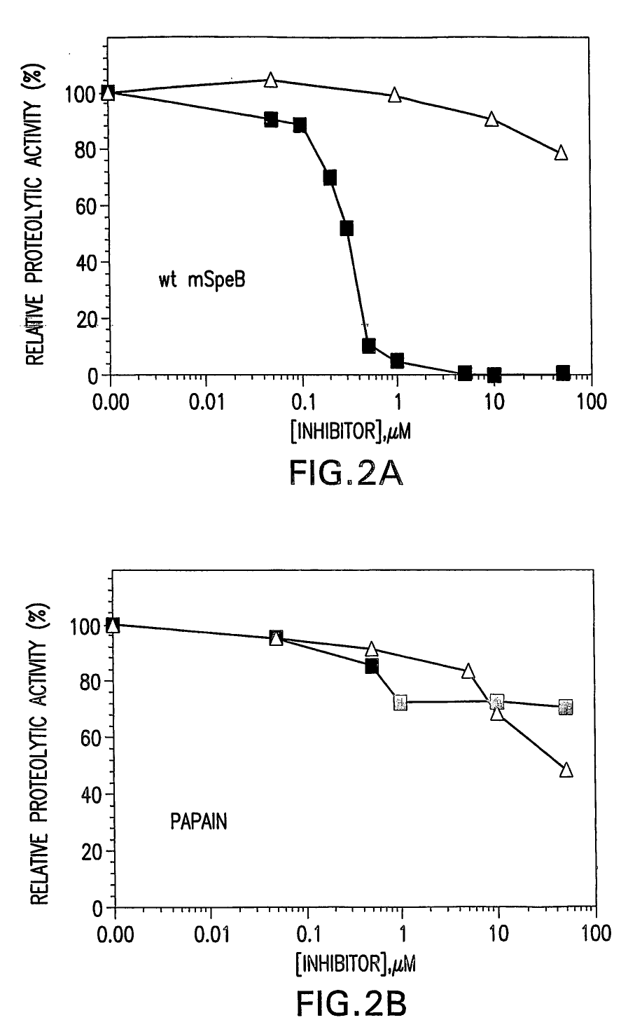

Association between the pro-sequence domain with the mature SpeB results in the inhibition of protease activity. This was demonstrated using a caseinolytic cleavage assay employing resorufin-labeled casein as a substrate (FIG. 2A). The pro-sequence domain, or a lysozyme control, was analyzed over a 0 to 100 μM inhibitor concentration range employing 0.1 μM of mature SpeB. Results indicated that the pro-sequence domain inhibited half the maximum protease activity at a concentration of 0.3 μM (IC₅₀=0.3 μM), while addition of lysozyme as an inhibitor had no effect on activity of the mature SpeB over the same concentration range. Analysis of a closely related cysteine protease (papain) under identical conditions displayed no effect on papain protease activity (FIG. 2B). Such results further demonstrate the specificity of interaction between the SpeB and the pro-sequence domain, and suggest that intramolecular inhibitory activity of the pro-sequence domain may serve to regulate protease activity in situ.

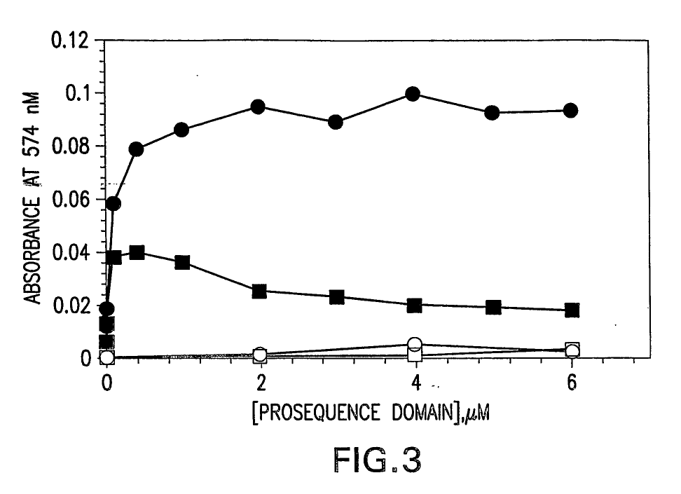

Intramolecular chaperone activity of the pro-sequence domain was demonstrated in vitro using urea-denatured mature Cysteine protease. Denatured mature protease refolded in the presence of increasing concentrations of pro-sequence domain, demonstrated a significant increase in recovered protease activity as monitored by the cleavage of a resorufin-labeled casein substrate (FIG. 3). Addition of an irreversible cysteine protease inhibitor (E-64) in the reaction prevented substrate cleavage, indicating that caseinolytic cleavage observed is specifically attributable to the activity of the refolded mature SpeB. These data suggest that the SpeB pro-sequence domain acts as an intramolecular chaperone to direct folding of the mature SpeB. Furthermore, the results demonstrate that the two regions need not be covalently linked to direct proper folding.

### Example 3

### Two-Plasmid Based Co-Expression of the SpeB Pro-Sequence Domain and Mature SpeB

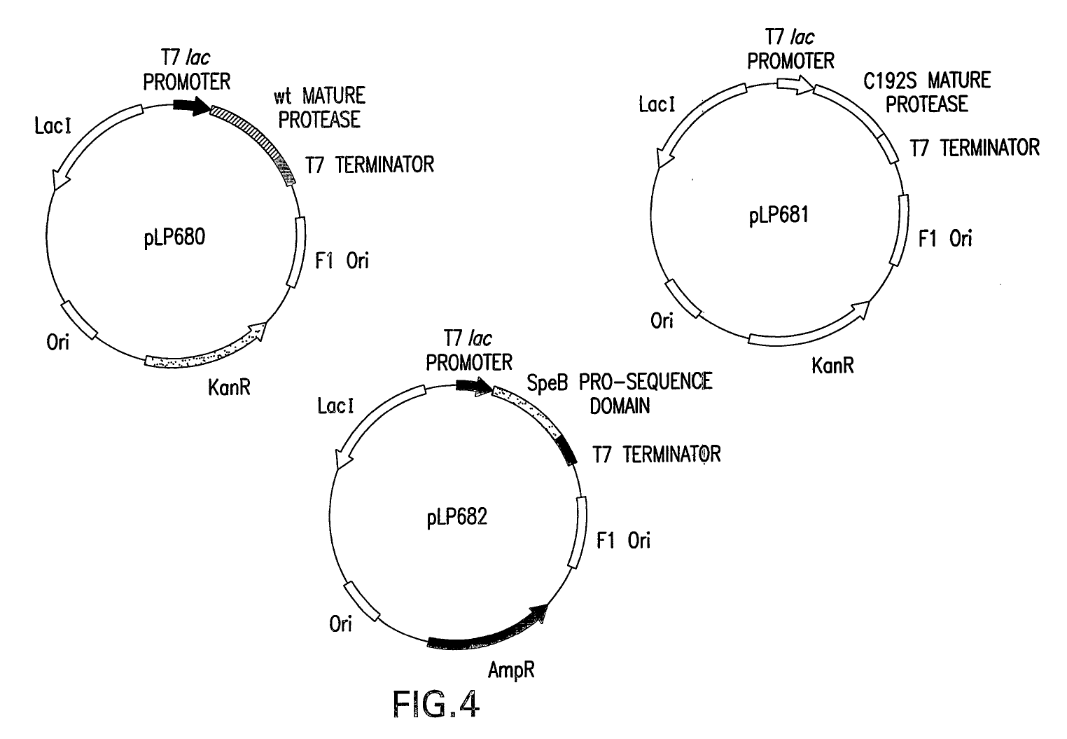

The ability of the pro-sequence domain to direct correct refolding of the mature SpeB polypeptide in vitro, suggests that independent co-expression of the two proteins in vivo would potentially result in the production of correctly folded mature SpeB. Thus, in this example, a two-plasmid co-expression system was developed where one plasmid encoded the pro-sequence domain (pLP682), and the other the mature SpeB polypeptide (pLP680 or pLP681) (FIG. 4). E. coli transformed with either pLP680 or pLP681 alone, or co-transformed with pLP682, were used to investigate protein expression using SDS-PAGE and Western blot. Sole expression of either the mature wild-type or mature C192S SpeB construct (data not shown) resulted in the production of predominantly insoluble 28 kDa mature SpeB. This suggests that expression of mature SpeB in the absence of the pro-sequence domain results in the production of incorrectly folded protein. In contrast, in vivo co-expression of the mature SpeB polypeptide in conjunction with the pro-sequence domain led to the production of substantial levels of both proteins in the soluble fraction of cells, indicating that the independent co-expression of both proteins promotes proper folding of the SpeB. Western blot analysis utilizing a polyclonal antibody directed against the SpeB zymogen allowed simultaneous detection of the pro-sequence domain and mature SpeB polypeptides (data not shown). Blot analysis confirmed the identity of the expressed proteins and further verified the results evidenced by SDS-PAGE. These results reconfirmed the in vitro data indicating that the pro-sequence domain and mature SpeB polypeptides need not be covalently linked to direct proper folding of the mature SpeB.

### Example 4

### Polycistronic Based Co-Expression of the SpeB Pro-Sequence Domain and Mature SpeB

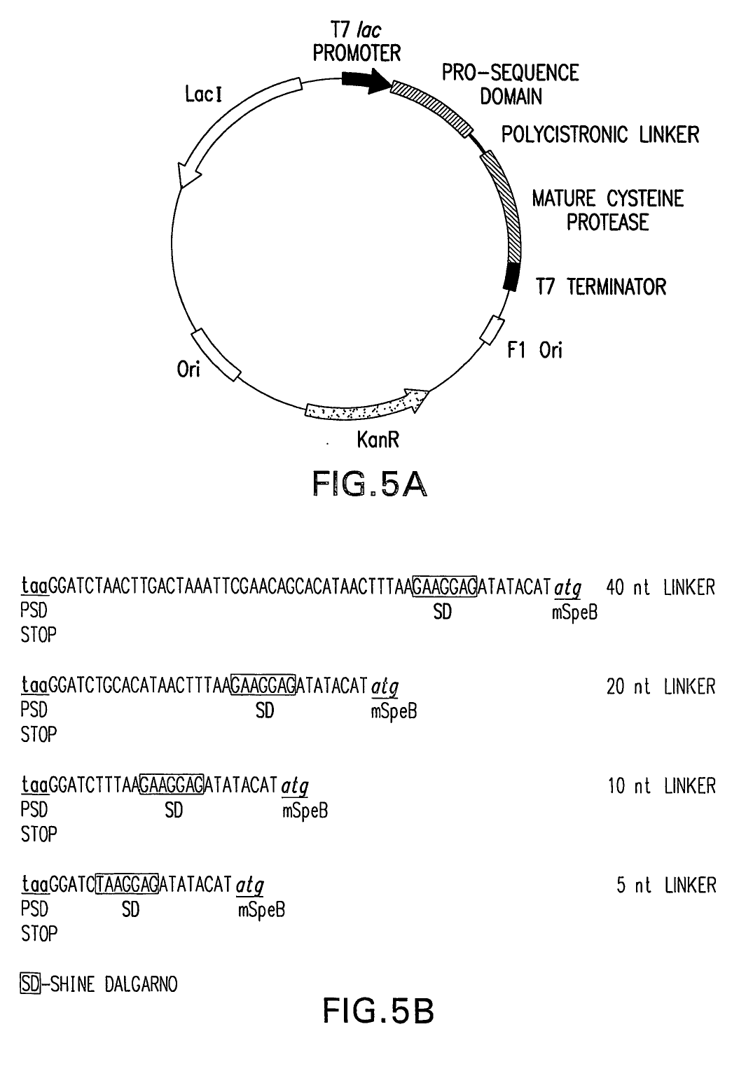

As described in Example 3, two-plasmid based co-expression of the pro-sequence domain and mature SpeB demonstrated the utility of such a method for the production of soluble, correctly folded SpeB. For large scale production of mature SpeB, a polycistronic expression system was developed for independent co-expression of the pro-sequence domain and mature SpeB polypeptides. Thus, the system was designed with the pro-sequence domain as the first cistron followed by a synthetic linker containing a translational enhancer and optimized Shine-Dalgarno ribosome binding site (Barrick et al., 1994; Curry and Tomich, 1998; Ringquist et al., 1992) 5′ to the second cistron (FIG. 5). Increasing lengths (5 nt, 10 nt, 20 nt, 40 nt) of synthetic linker between translational stop codon of the first cistron and Shine-Dalgarno of the second cistron were investigated for differences in expression levels of the two proteins. Linker regions were designed to minimize secondary structure between the two cistrons in the transcribed RNA, allowing for unrestricted ribosome flow and efficient re-initiation of translation at the second cistron. Experiments utilizing polycistronic mature C192S SpeB expression constructs pLP683, pLP684, pLP685, and pLP686 containing the 5 nt, 10 nt, 20 nt, and 40 nt linkers, respectively, as well as the 20 nt polycistronic expression construct for mature wild-type SpeB (pLP687), were performed.

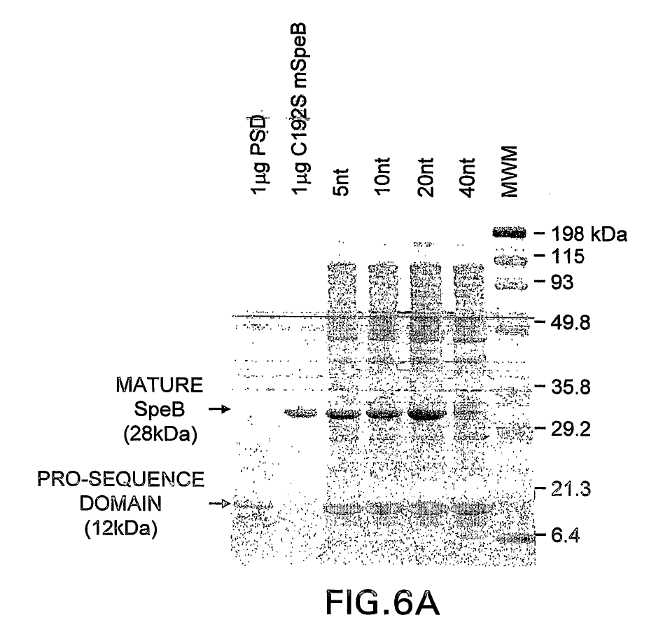

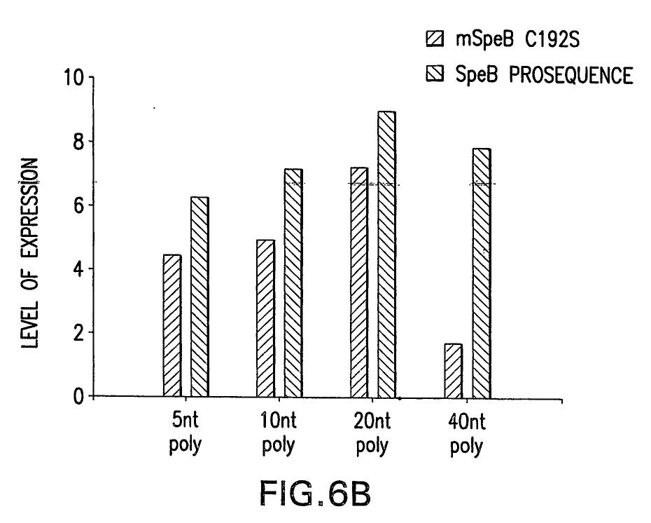

Analysis of protein expression using the pLP685 and pLP687 polycistronic constructs confirmed the production of both the pro-sequence domain (12 kDa) and mature SpeB (28 kDa) (data not shown). As demonstrated with the two-plasmid system, simultaneous and independent co-expression of both proteins in E. coli resulted in the production of mature SpeB predominantly in the soluble fraction of the cells. Soluble fractions of whole cell lysates from induced cultures of each of the four C192S polycistronic constructs were also analyzed by SDS-PAGE (FIG. 6A). To quantitate the levels of expression, the gel was analyzed by use of a densitometer and the area of each band corresponding to the 28 kDa mature SpeB, or the 12 kDa pro-sequence domain, was measured (FIG. 6B). Although little difference was observed in the level of pro-sequence domain expressed by each construct, a marked decrease in soluble mature SpeB expression from the 40 nt linker-containing polycistron was observed. Interestingly, soluble mature SpeB expression from a construct containing a 119 nt linker region demonstrated levels comparable to that of the 5 nt, 10 nt, and 20 nt linker polycistrons (data not shown).

### Example 5

### Analysis of Polycistronic mRNA Transcripts and Evaluation of Transcriptional Levels

Total RNA was isolated from induced cultures of all polycistronic constructs, the wild-type SpeB zymogen, mature wild-type SpeB, mature C192S SpeB, and pro-sequence domain. Northern blot analysis of isolated RNA was performed to assess the size of transcripts produced from the 20 nt linker-containing constructs, as well as the appropriate positive controls as indicated (data not shown). Results obtained demonstrated that mature wild-type SpeB (˜892 bases), mature C192S SpeB (˜892 bases), and pro-sequence domain (˜487 bases) mRNA transcripts from controls all migrated at expected sizes. Detection of mRNA transcripts from both the wild-type and C192S 20 nt linker containing polycistronic expression systems revealed transcript signals migrating at an equivalent size (˜1287 bases), slightly higher than that for the wild-type SpeB zymogen control (˜1246 bases). Such results are consistent with the generation of a full-length, polycistronic mRNA for each, as predicted. The lack of detection of smaller transcripts in these samples, equivalent to those seen for the mature SpeB and pro-sequence domain controls, indicates that expression of the SpeB is directly attributable to polycistronic transcript production and not the result of the presence of multiple mRNA species.

The full-length nature of transcripts produced from all polycistronic species was also verified by PCR analysis of cDNA. Total RNA from polycistronic samples and single cistron controls was used to produce the corresponding cDNA, and diluted cDNA samples were analyzed by PCR. A negative control for each sample, which contained all reaction components except the reverse transcriptase (−RT), was generated to assess potential plasmid DNA contamination within isolated RNA. Primer sets specific for the pro-sequence domain (367 bp), mature SpeB (770 bp), and full length pro-sequence/mature SpeB (1119-1180 bp) coding regions were used to examine cDNA samples, —RT negative controls, and a pLP685 positive control (data not shown). PCR amplification of the pro-sequence domain produced products of the expected size in all cDNA samples containing a pro-sequence domain nucleotide sequence in their expression constructs. Samples lacking this sequence, the mature wild-type SpeB and mature C192S SpeB expression constructs, failed to produce amplification products. For amplification of the mature protease coding region, all cDNA samples except that of the pro-sequence domain expression system produced positive bands. Amplification of the full-length pro-sequence/mature SpeB region produced PCR products for only the polycistronic and SpeB zymogen samples, while samples from the mature wild-type SpeB, mature C192S SpeB and pro-sequence domain expression systems evidenced lack of product generation, as predicted. The -RT negative controls for all samples produced no amplification products, indicating that the production of positive signals observed in cDNA samples originated from reverse transcription of mRNA, not amplification of contaminating DNA.

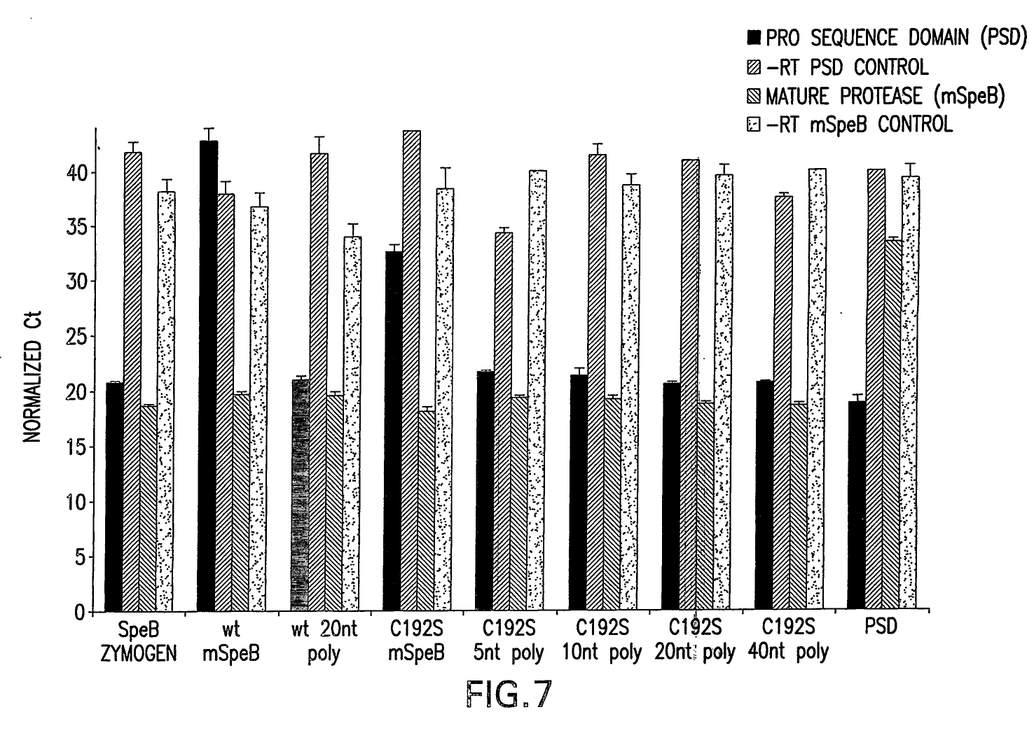

Examination of mRNA transcription levels for the different expression systems was determined by quantitative PCR analysis of cDNA samples and −RT controls. Primer/probe sets specific for each of the pro-sequence and mature SpeB regions were used to compare transcript levels between cultures. Given that the gene encoding kanamycin resistance is present on all expression constructs, KanR mRNA was used as an internal standard during analysis to control for potential differences in plasmid copy number between expression samples. The results demonstrate that the levels of mRNA transcripts generated during induction of each construct are equivalent (FIG. 7). More importantly, the data indicate that mRNA levels for the pro-sequence is comparable to that of the mature SpeB. These results suggest that apparent differences in the observed amounts of the soluble mature SpeB between the polycistronic constructs are not due to premature termination of transcription.

### Example 6

### Characterization of Co-Expressed Mature SpeB

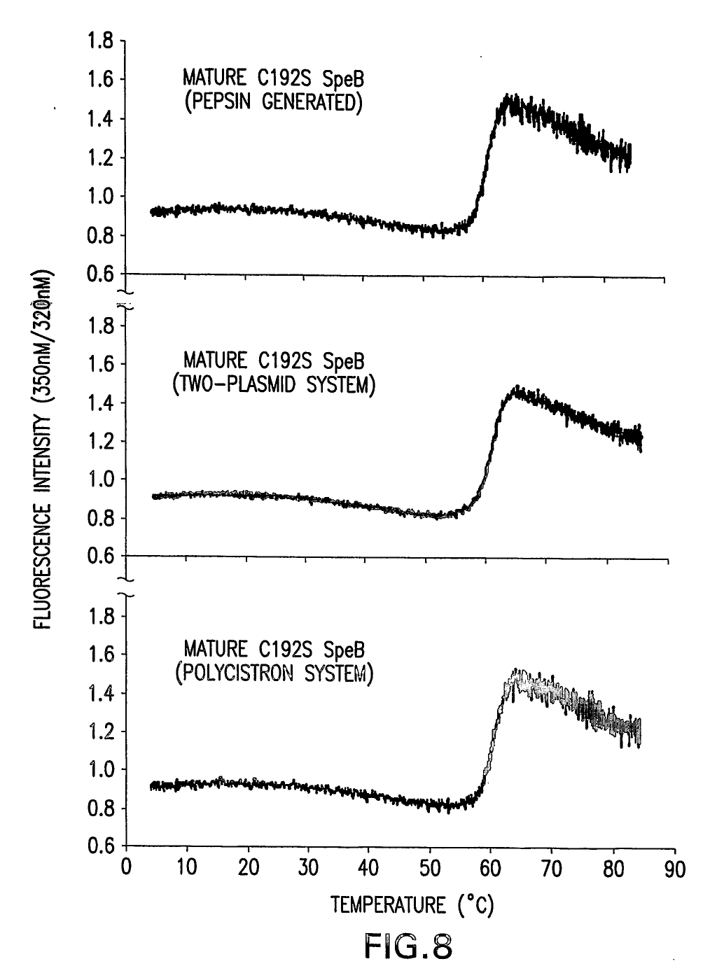

The purified mature SpeB generated by both two-plasmid based and polycistronic systems were analyzed by SDS-PAGE (data not shown), and all proteins were subjected to heat-induced unfolding experiments (FIG. 8 and FIG. 9). Melting (i.e., denaturation) curves were obtained by heating protein samples over an increasing temperature range (0-90° C.), while monitoring the change in the ratio of intrinsic fluorescence. Denaturation curves generated for both the two-plasmid and polycistronic co-expressed C192S (FIG. 8), or wild-type (FIG. 9) mature SpeB demonstrated well-defined transitions with midpoints similar to that of their respective counterparts expressed as SpeB zymogens and processed by in vitro cleavage (papain generated), or autocatalytically (wild-type SpeB zymogen). Such results indicate that the recombinant proteins produced by either co-expression system are folded similarly to their corresponding recombinantly expressed and processed zymogens.

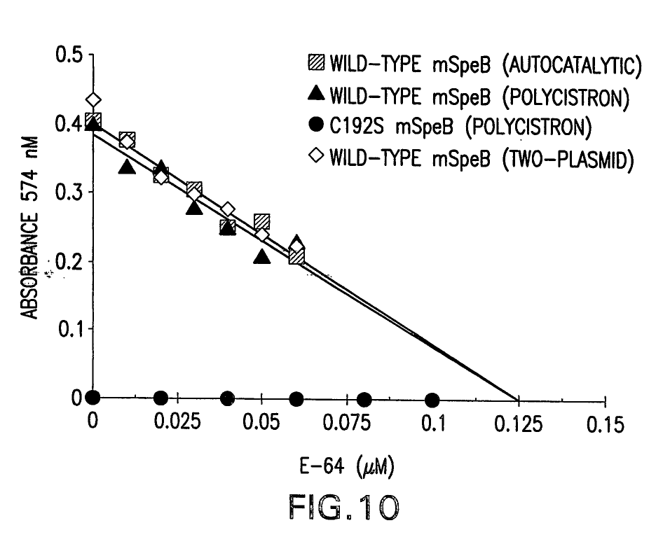

Inherent protease activities of all recombinant mature wild-type SpeB were evaluated by determination of their operational molarity (FIG. 10). For each, equivalent concentrations of SpeB (0.12 μM) were pre-incubated in the presence of increasing amounts of the irreversible cysteine protease inhibitor, E-64, prior to addition of a resorufin-labeled casein substrate. The release of labeled peptides was measured spectrophotometrically and results plotted as a function of inhibitor concentration. Values obtained for the two-plasmid (0.121 μM), polycistronic (0.124 μM) and autocatalytically processed (0.124 μM) mature SpeB polypeptides indicated that they are enzymatically indistinguishable and are equivalent to the expected value of 0.12 μM based upon protein concentration.

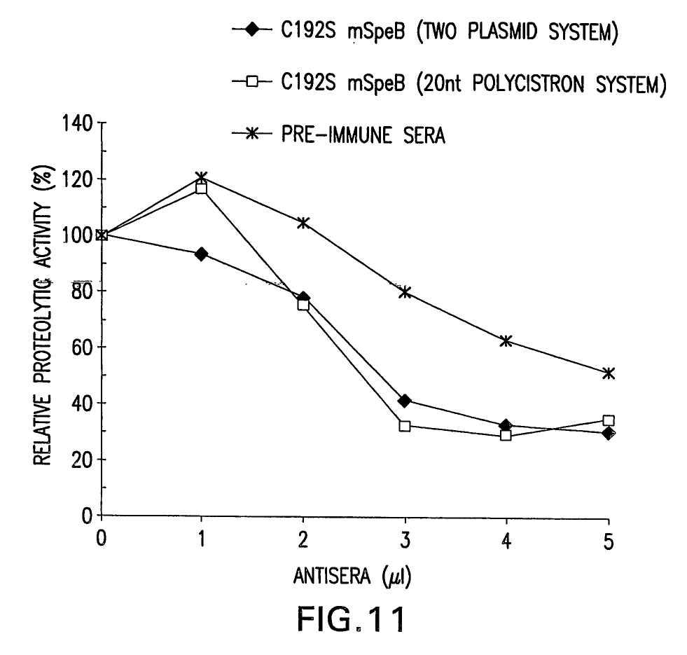

Previous data indicated that antibodies generated against mature C192S SpeB produced by in vitro cleavage with papain were capable of inhibiting proteolytic activity of wild-type SpeB (Matsul<a et al., 1999). To determine whether recombinant protein produced via either co-expression system was capable of eliciting similar activity, increasing concentrations of antiserum from mice immunized with mature C192S SpeB generated by either the two-plasmid or 20 nt polycistronic system were analyzed using caseinolytic cleavage assay (FIG. 11). Results indicate that hydrolysis of substrate is inhibited in the presence of increasing amounts of serum from animals immunized with protein produced by either co-expression system relative to the pre-immune control. Furthermore, the levels of inhibition evidenced for each are comparable to those previously reported for serum generated against mature C192S SpeB expressed as a SpeB zymogen and processed in vitro using pepsin (Matsuka et al., 1999).

### References

International Application No. EP 125,023
International Application No. EP 171,496
International Application No. EP 184,187
International Application No. EP 173,494
International Application No. EP 264166
International Application No. PCT/US86/02269
International Application No. WO 86/01533
International Application No. WO 91/17271
International Application No. WO 92/01047
International Application No. WO 92/09690
International Application No. WO 92/15679
International Application No. WO 92/18619
International Application No. WO 92/20791
International Application No. WO 93/01288
International Application No. WO 00/63364
International Application No. WO 94/12650
International Application No. WO 93/13302
International Application No. WO 92/19265
International Application No. WO 00/18434
International Application No. WO 93/13202
International Application No. WO 96/05858
International Application No. WO 98/20734
International Patent No. WO 98/42375
U.S. Pat. No. 4,196,265
U.S. Pat. No. 4,816,567
U.S. Pat. No. 4,873,316
U.S. Pat. No. 4,987,071
U.S. Pat. No. 4,912,094
U.S. Pat. No. 4,837,151
U.S. Pat. No. 4,522,811
U.S. Pat. No. 4,554,101
U.S. Pat. No. 4,683,202
U.S. Pat. No. 4,987,071
U.S. Pat. No. 5,968,502
U.S. Pat. No. 5,223,409
U.S. Pat. No. 5,116,742
U.S. Pat. No. 5,723,127
U.S. Pat. No. 5,643,576.
U.S. Pat. No. 5,593,972
U.S. Pat. No. 5,580,859
U.S. Pat. No. 5,168,062
U.S. Pat. No. 5,078,996
U.S. Pat. No. 5,057,540
U.S. Pat. No. 5,116,742
U.S. Pat. No. 5,108,744
U.S. Pat. No. 5,593,972
U.S. Pat. No. 5,601,831
U.S. Pat. No. 5,614,382
U.S. Pat. No. 5,785,973
U.S. Pat. No. 6,207,646
U.S. Pat. No. 6,168,918
U.S. Pat. No. 6,127,170
U.S. Pat. No. 6,113,918
Amann et al., Gene 69:301-315, 1988.
Anonymous, “Consensus”, Pediatr. Infect. Dis. J., 8:594-597,1989.
Ausubel et al., 1995, Current Protocols in Molecular Biology, eds., John Wiley & Sons, Inc., sections 2.10 and 6.3-6.4
Baldari et al., Embo J. 6:229-234, 1987.
Banerji et al., Cell, 33:729-740; 1983.
Barany et al., Int. J. Peptide Protein Res. 30, 705-739, 1987.
Barrick et al., Nucl. Acids Res., 22:1287-1295, 1994.
Bennett, Haggard, Silva, & Stewart, “Behavior and developmental effects of otitis media with effusion into the teens,” Arch. Dis. Child., 85, 91-95, 2001.
Bootsma, van der Heide, van de Pas, Schouls, & Mooi, “Analysis of Moraxella catarrhalis by DNA typing: evidence for a distinct subpopulation associated with virulence traits,” J. Infect. Dis., 181,1376-1387, 2000.
Byrne and Ruddle, PNAS 86:5473-5477, 1989.
Calame and Eaton, Adv. Immunol. 43:235-275, 1988.
Campagnari, Shanks & Dyer, “Growth of Moraxella catarrhalis with human transferrin and lactoferrin: expression of iron-repressible proteins without siderophore production,” Infection and Immunity, 62(11), 4909-4914, 1994.
Campes and Tilghman, Genes Dev. 3:537-546, 1989.
Carson, Klebba, Newton, & Sparling, “Ferric enterobactin binding and utilization by Neisseria gonorrhoeae,” J. Bacteriol., 181(9), 2895-2901, 1999.
Chaussee et al., Infect. Immun.; 61:3719-3723, 1993.
Chen et al., “Evaluation of purified UspA from Moraxella catarrhalis as a vaccine in a murine model after active immunization,” Infect. Immun., 64(6), 1900-1905, 1996.
Creighton, T. E., “PROTEINS, Structures and Molecular Properties”, 2^{nd} Ed., W.H. Freedman and Co., NY, 1993.
Cunningham, “Pathogenesis of group A streptococcal infections,” Clin. Microbiol. Rev., 13:470-511, 2000.
Curry and Tomich, DNA; 7:173-179, 1988.
Davies et al., “Invasive Group A streptococcal infections in Ontario, Canada,” N. Eng. J. Med., 335:547-554, 1996.
de Velasco et al., “Synthetic peptides representing T-cell epitopes act as carriers in pneumococcal polysaccharide conjugate vaccines,” Infect. Immun., 63:961-968, 1995.
Edlund et al., Science 230:912-916, 1985.
Frohman et al., Proc. Natl. Acad. Sci. USA 85, 8998-9002, 1988.
Gaultier et al., Nucleic Acids Res. 15:6625-6641, 1987.
Gentz et al., Proc. Natl. Acad. Sci. USA, 86:821-824, 1989.
Gray, Conjugate Vaccines Supplement p 694-697, 1990.
Gubba et al., Infect Immun.; 66:765-770, 1998.
Harlow and Lane, “Antibodies: A Laboratory Manual,” Cold Spring Harbor Laboratory Press, Cold Spring Harbor, N.Y., 1988.
Hartl and Hayer-Hartl, “Molecular Chaperones in the Cytosol: from Nascent Chain to Folded Protein”, Science, 295:1852-1858, 2002.
Harrington et al., Nature Biotechnology, 19:440-445, 2001.
Haselhoff and Gerlach, Nature 334:585-591, 1988.
Helene et al., Ann. N. Y Acad Sci. 660:27-36,1992.
Helene, Anticancer Drug Des. 6(6):569-84, 1991.
Helminen et al., ”A mutation affecting expression of a major outer membrane protein of Moraxella catarrhalis alters serum resistance and survival in vivo,“J. Infect. Dis., 168,1194-1201, 1993a.
Helminen, Maciver, Latimer, Cope, McCracken, & Hansen, “A major outer membrane protein of Moraxella catarrhalis is a target for antibodies that enhance pulmonary clearance of the pathogen in an animal model,” Infect Immun, 61(5), 2003-2010,1993b.
Henriksen et al., “Vaccination with protein-conjugated and native type 3 capsular polysaccharide in an ethanol-fed rat model of pneumococcal pneumonia”, Alcohol Clin. Exp. Res., 21:1630-1637,1997. in 2000, a state-of-the-art review.” Clin. Microbiol. Rev., 14(2), 336-363, 2001.
Hope-Simpson, “Streptococcus pyogenes in the throat: A study in a small population, 1962-1975” J. Hyg. Camb., 87:109-129, 1981.
Inoue et al., FEBS Lett. 215:327-330, 1987(a).
Inoue et al., Nucleic Acids Res. 15:6131-6148, 1987(b).
Johnson et al., Endoc. Rev., 10:317-331, 1989.
Kaufman et al., EMBO J 6:187-195, 1987.
Kessel and Gruss, Science 249:374-379, 1990.
Khan and James, Protein Sci., 7:815-836, 1998.
Kurj an and Herskowitz, Cell 933-943, 1982.
Kyte and Doolittle, J. Mol. Biol., 157:105-132, 1982.
Lafontaine, Cope, Aebi, Latimer, McCracken, & Hansen, “The UspA1 protein and a second type of UspA2 protein mediate adherence of Moraxella catarrhalis to human epithelial cells in vitro,” Bacteriol., 182(5), 1364-1373, 2000.
Liu and Elliot, J. Biol. Chem., 240:1138-1142, 1965.
Liu et al., J. Biol. Chem., 240:1143-1149, 1965.
Liu, J. Biol Chem., 242:4029-4032, 1965.
Lorimer and Baldwin, Methods in Enzymology, Vol. 290, Molecular Chaperones, Academic Press, NY, 1998.
Lucklow and Summers, Virology 170:31-39, 1989.
Lyon et al., “A Role for Trigger Factor and an Rgg-like regulator in the transcription, secretion and processing of the cysteine proteinase of Streptococcus pyogenes,” EMBO J., 17:6263-6275, 1998.
Maher, Bioassays 14(12):807-15, 1992.
Matsuka et al., Infect. Immun., 67:4326-4333, 1999.
Murphy, “Branhamella catarrhalis: epidemiology, surface antigenic structure, and immune response,” Microbiol. Rev., 60, 267-279, 1996.
Musser et al., Infect. Immun., 64:1913-1917, 1996.
Pinkert et al. Genes Dev. 1:268-277, 1987.
Queen and Baltimore, Cell 33:741-748, 1983.
Ringquist et al., Mol. Microbiol.; 6:1219-1229, 1992.
Sambrook et al., “Molecular Cloning: A Laboratory Manual” 2nd ed, Cold Spring Harbor Laboratory, Cold Spring Harbor Laboratory Press, Cold Spring Harbor, N.Y., 1989
Sambrook et al., “Molecular Cloning: A Laboratory Manual” 2nd, ed, Cold Spring Harbor Laboratory, Cold Spring Harbor Laboratory Press, Cold Spring Harbor, N.Y., 1989.
Sasaki, Harkness, Loosmore, Chong, & Klein, “High molecular weight major outer membrane protein of Moraxella,” in Patent Aventis, United States, 2001.
Schultz et al., Gene 54:113-123, 1987
Seed, Nature 329:840, 1987.
Sethi & Murphy, “Bacterial infection in chronic obstructive pulmonary disease
Smith and Johnson, Gene 67:31-40, 1988.
Smith et al., Anal. Biochem.,1 50:76-85, 1985.
Smith et al., Mol. Cell Biol. 3:2156-2165, 1983.
Studier et al. ”Gene Expression Technology” Methods in Enzymology 185, 60-89, 1990.
Stevens, “Invasive group A streptococcus infections,” Clin. Infect. Dis., 14:2-13, 1992.
Stevens et al., “Penicillin Binding Protein Expression at Different Growth Stages Determines Penicillin Efficacy In Vitro an In Vivo: An Explanation of the Inoculum Effect,” J. Infect. Disease, 167:1401-1405, 1993.
Tai et al., J. Biol. Chem., 251:1955-1959, 1976.
Tucker, Plosila & Tillman, “Moraxella catarrhalis outer membrane protein-1 06 polypeptide, gene sequence and uses thereof. In U.S. Patent, Antex Biologics Inc., United States, 2001.
Weiner et al., Gene; 151:119-123, 1994.
Winoto and Baltimore, EMBO J, 8:729-733, 1989.
Betton, “Rapid Translation System (RTS): A Promising Alternative for Recombinant Protein Production,” Curr. Protein Pept. Sci., 4(1):73-80, 2003.
Braun et al., “Proteome-scale purification of human proteins from bacteria,” Proc. Natl. Acad. Sci. U.S.A., 99(5):2654-9, 2002.
Jermutus et al., “Recent advances in producing and selecting functional proteins by using cell-free translation,” Curr. Opin. Biotechnol., 9; 534-548, 1998.
Kigawa et al., “Cell-free production and stable-isotope labeling of milligram quantities of proteins,” FEBS Lett.; 442(1):15-19, 1999.
Kim et al., “A highly efficient cell-free protein synthesis system from Escherichia coli,” Eur. J. Biochem., 239(3):881-886, 1996.
Spirin et al., “A continuous cell-free translation system capable of producing polypeptides in high yield,” Science, 242:1162-1164, 1998.
Betton, Braun et al, Jermutus et al., Kigawa et al., Kim et al., Spirin et al.,

What is claimed is:

1. A method for recombinantly expressing a mature Streptococcus pyogenes exotoxin B (SpeB) polypeptide in a host cell, the method comprising transforming, transducing, transfecting or infecting a host cell with a polycistronic plasmid, the plasmid comprising (a) a polynucleotide sequence encoding a SpeB pro-polypeptide domain and (b) a polynucleotide sequence encoding a mature SpeB polypeptide, and culturing the host cell under conditions which permit the expression of the mature SpeB polypeptide and the SpeB pro-polypeptide domain by the host cell, and wherein the mature SpeB polypeptide is soluble in the host cell.

2. The method of claim 1, wherein the SpeB pro-polypeptide domain is further defined as a polypeptide comprising amino acid residues 28 through 145 of SEQ ID NO:2.

3. The method of claim 1, wherein the mature SpeB polypeptide is further defined as a polypeptide comprising amino acid residues 146 through 398 of SEQ ID NO:2.

4. The method of claim 1, wherein the cysteine at amino acid residue 192 of the mature SpeB polypeptide is substituted by a serine.

5. The method of claim 1, wherein the mature SpeB polypeptide is immunogenic in a mammalian host.

6. The method of claim 1, wherein an antibody specific for the mature SpeB polypeptide cross-reacts with a wild-type SpeB polypeptide and neutralizes SpeB polypeptide activity.

7. The method of claim 1, wherein the plasmid is a T7 promoter-containing plasmid.

8. The method of claim 7, wherein the plasmid is selected from the group consisting of pET, pRSET, pCRT7-CTTOPO and pIVeX.

9. The method of claim 1, wherein the host cell is a bacterial cell.

10. The method of claim 9, wherein the host cell is E. coli.

11. The method of claim 10, wherein the E. coli is a strain selected from the group consisting of BLR(DE3), BLR(DE3)pLysS, AD494(DE3), AD494(DE3)pLysS, BL21(DE3), BL21(DE3) pLysS, BL21(DE3)pLysE, BL21(DE3)pLacI, BL21trxB(DE3), BL21trxB(DE3)pLysS, HMS174(DE3), HMS174(DE3)pLysS, HMS174(DE3)pLysE, Origami(DE3), Origami(DE3)pLysS, Origami(DE3)pLysE, Origami(DE3)pLacI, OrigamiB(DE3), OrigamiB(DE3)pLysS, OrigamiB(DE3)pLysE, OrigamiB(DE3)pLacI, Rosetta(DE3), Rosetta(DE3)pLysS, Rosetta(DE3)pLysE, Rosetta(DE3)pLacI, Tuner(DE3), Tuner(DE3)pLysS and Tuner(DE3)pLacI.

12. A method for recombinantly expressing a mature SpeB polypeptide in a host cell, the method comprising:
(a) transforming, transducing, transfecting or infecting a host cell with (i) a plasmid comprising a polynucleotide sequence encoding a SpeB pro-polypeptide domain and (ii) a plasmid comprising a polynucleotide sequence encoding a mature SpeB polypeptide; and
(b) culturing the host cell under conditions suitable to co-express the SpeB pro-polypeptide domain and the mature SpeB polypeptide,
wherein the mature SpeB polypeptide is soluble in the host cell.

13. The method of claim 12, wherein the SpeB pro-polypeptide domain is further defined as a polypeptide comprising amino acid residues 28 through 145 of SEQ ID NO:2.

14. The method of claim 12, wherein the mature SpeB polypeptide is further defined as a polypeptide comprising amino acid residues 146 through 398 of SEQ ID NO:2.

15. The method of claim 12, wherein the cysteine at amino acid residue 192 of the mature SpeB polypeptide is substituted by a serine.

16. The method of claim 12, wherein the mature SpeB polypeptide is immunogenic in a mammalian host.

17. The method of claim 12, wherein an antibody specific for the mature SpeB polypeptide cross-reacts with a wild-type SpeB polypeptide and neutralizes SpeB polypeptide activity.

18. The method of claim 12, wherein the plasmid is a T7 promoter-containing plasmid.

19. The method of claim 18, wherein the plasmid is selected from the group consisting of pET, pRSET, pCRT7-CTTOPO and pIVeX.

20. The method of claim 12, wherein the host cell is a bacterial cell.

21. The method of claim 20, wherein the host cell is E. col.

22. The method of claim 21, wherein the E. coli is a strain selected from the group consisting of BLR(DE3), BLR(DE3)pLysS, AD494(DE3), AD494(DE3)pLysS, BL21(DE3), BL21(DE3) pLysS, BL21(DE3)pLysE, BL21(DE3)pLacI, BL21trxB(DE3), B21trxB(DE3)pLysS, HMS174(DE3), HMS174(DE3)pLysS, HMS174(DE3)pLysE, Origami(DE3), Origami(DE3)pLysS, Origami(DE3)pLysE, Origami(DE3)pLacI, OrigamiB(DE3), OrigamiB(DE3)pLysS, OrigamiB(DE3)pLysE, OrigamiB(DE3)pLacI, Rosetta(DE3), Rosetta(DE3)pLysS, Rosetta(DE3)pLysE, Rosetta(DE3)pLacI, Tuner(DE3), Tuner(DE3)pLysS and Tuner(DE3)pLacI.

23. A method for producing a mature SpeB polypeptide comprising the steps of:
(a) recombinantly expressing in a host cell a plasmid comprising a polynucleotide sequence encoding a mature SpeB polypeptide, wherein the SpeB polypeptide forms an insoluble polypeptide aggregate in the host cell;
(b) solubilizing the polypeptide aggregate, wherein the solubilized polypeptide is defined as a non-native mature SpeB polypeptide;
(c) refolding the non-native mature SpeB polypeptide in the presence of one or more chaperone proteins, wherein the non-native mature SpeB polypeptide is folded into a native mature SpeB polypeptide; and
(d) recovering the native mature SpeB polypeptide.

24. The method of claim 23, wherein the one or more chaperone proteins are selected from the group consisting of GroEL, GroEUGroES, peptidyl-prolyl isomerase (PPI), peptide disulfide isomerase (PDI) and a SpeB pro-polypeptide domain.

25. The method of claim 23, wherein the chaperone protein is a SpeB pro-polypeptide domain comprising amino acid residues 28 through 145 of SEQ ID NO:2.

26. The method of claim 23, wherein the mature SpeB is a polypeptide comprising amino acid residues 146 through 398 of SEQ ID NO:2.

27. The method of claim 26, wherein the cysteine at amino acid residue 192 of the mature SpeB polypeptide is substituted by a serine.

28. The method of claim 23, wherein the insoluble polypeptide aggregate is further defined as an inclusion body.

29. The method of claim 23, wherein solubilizing the polypeptide is a denaturant selected from the group consisting of urea, guanidinium chloride and heat.

30. A method for recombinantly expressing a mature SpeB polypeptide in a host cell comprising expressing in a host cell a polycistronic plasmid comprising (i) a polynucleotide sequence encoding a mature SpeB polypeptide and (ii) a polynucleotide sequence encoding a GroEL polypeptide, wherein the mature SpeB polypeptide is soluble in the host cell.

31. The method of claim 30, wherein the cysteine at amino acid residue 192 of the mature SpeB polypeptide is substituted by a serine.

32. The method of claim 30, wherein the plasmid further comprises a polynucleotide encoding a GroES polypeptide.

33. A method for producing a mature SpeB polypeptide comprising the steps of:
(a) transforming, transducing, transfecting or infecting a host cell with a polycistronic plasmid comprising (i) a polynucleotide sequence encoding a mature SpeB polypeptide and (ii) a polynucleotide sequence encoding a GroEL polypeptide;
(b) culturing the host cell under conditions suitable to express the mature SpeB polypeptide and the GroEL polypeptide, wherein the mature SpeB polypeptide is soluble in the host cell; and
(c) recovering the native mature SpeB polypeptide.

34. The method of claim 33, wherein the cysteine at amino acid residue 192 of the mature SpeB polypeptide is substituted by a serine.

35. A mature SpeB polypeptide produced according to the method of claim 1.

36. A mature SpeB polypeptide produced according to the method of claim 12.

37. A mature SpeB polypeptide produced according to the method of claim 23.

38. A mature SpeB polypeptide produced according to the method of claim 30.

39. A mature SpeB polypeptide produced according to the method of claim 33.

40. An immunogenic composition comprising the SpeB polypeptide of claim 35.

41. An immunogenic composition comprising the SpeB polypeptide of claim 36.

42. An immunogenic composition comprising the SpeB polypeptide of claim 37.

43. An immunogenic composition comprising the SpeB polypeptide of claim 38.

44. An immunogenic composition comprising the SpeB polypeptide of claim 39.

45. A method of immunizing a mammalian subject against S. pyogenes comprising administering to the subject an immunogenic amount of the composition of claim 40.

46. A method of immunizing a mammalian subject against S. pyogenes comprising administering to the subject an immunogenic amount of the composition of claim 41.

47. A method of immunizing a mammalian subject against S. pyogenes comprising administering to the subject an immunogenic amount of the composition of claim 42.

48. A method of immunizing a mammalian subject against S. pyogenes comprising administering to the subject an immunogenic amount of the composition of claim 43.

49. A method of immunizing a mammalian subject against S. pyogenes comprising administering to the subject an immunogenic amount of the composition of claim 44.

50. A polycistronic plasmid comprising (a) a polynucleotide sequence encoding a SpeB pro-polypeptide domain and (b) a polynucleotide sequence encoding a mature SpeB polypeptide, wherein the mature SpeB polypeptide is soluble when expressed in a host cell.

51. The plasmid of claim 50, wherein the cysteine at amino acid residue 192 of the mature SpeB polypeptide is substituted by a serine.

52. The plasmid of claim 50, wherein the plasmid is a T7 promoter-containing plasmid.

53. A plasmid comprising a polynucleotide sequence encoding a SpeB pro-polypeptide domain and a plasmid comprising a polynucleotide sequence encoding a mature SpeB polypeptide, wherein the mature SpeB polypeptide is soluble when expressed in a host cell.

54. The plasmid of claim 53, wherein the cysteine at amino acid residue 192 of the mature SpeB polypeptide is substituted by a serine.

55. The plasmid of claim 53, wherein the plasmid is a T7 promoter-containing plasmid.

56. A polycistronic plasmid comprising (a) a polynucleotide sequence encoding a mature SpeB polypeptide and (b) a polynucleotide sequence encoding a GroEL polypeptide, wherein the mature SpeB polypeptide is soluble when expressed in a host cell.

57. The plasmid of claim 56, wherein the cysteine at amino acid residue 192 of the mature SpeB polypeptide is substituted by a serine.

58. The plasmid of claim 56, wherein the plasmid is a T7 promoter-containing plasmid.

59. A polycistronic plasmid comprising (a) a polynucleotide sequence encoding a mature SpeB polypeptide, (b) a polynucleotide sequence encoding a GroEL polypeptide and (c) a polynucleotide sequence encoding a GroES polypeptide, wherein the mature SpeB polypeptide is soluble when expressed in a host cell.

60. The plasmid of claim 59, wherein the cysteine at amino acid residue 192 of the mature SpeB polypeptide is substituted by a serine.

61. The plasmid of claim 59, wherein the plasmid is a T7 promoter-containing plasmid.

62. A polycistronic plasmid comprising (a) a polynucleotide sequence encoding a mature SpeB polypeptide and (b) a polynucleotide sequence encoding one or more polypeptides selected from the group consisting of GroEL, GroES, SpeB pro-polypeptide domain, PDI and PPI, wherein the mature SpeB polypeptide is soluble when expressed in a host cell.

63. The plasmid of claim 62, wherein the cysteine at amino acid residue 192 of the mature SpeB polypeptide is substituted by a serine.

64. The plasmid of claim 62, wherein the plasmid is a T7 promoter-containing plasmid.

65. A host cell transformed, transduced, transfected or infected with the plasmid of claim 50.

66. A host cell transformed, transduced, transfected or infected with the plasmid of claim 53.

67. A host cell transformed, transduced, transfected or infected with the plasmid of claim 56.

68. A host cell transformed, transduced, transfected or infected with the plasmid of claim 59.

69. A host cell transformed, transduced, transfected or infected with the plasmid of claim 62.

70. The method of claim 24, wherein the PPI is a S. pyogenes RopA PPI.

71. The plasmid of claim 62, wherein the PPI is a S. pyogenes RopA PPI.


SEQUENCE LISTING (15 sequences)

SEQ ID NO: 1 - DNA, 1197 bp, Streptococcus pyogenes
atgaataaaaagaaattaggtgtcagattattaagtcttttagcattaggtggatttgttcttgctaacccagtatttgccgatcaaaactttgctcgtaacgaaaaagaagcaaaagatagcgctatcacatttatccaaaaatcagcagctatcaaagcaggtgcacgaagcgcagaagatattaagcttgacaaagttaacttaggtggagaactttctggctctaatatgtatgtttacaatatttctactggaggatttgttatcgtttcaggagataaacgttctccagaaattctaggatactctaccagcggatcatttgacgctaacggtaaagaaaacattgcttccttcatggaaagttatgtcgaacaaatcaaagaaaacaaaaaattagacactacttatgctggtaccgctgagattaaacaaccagttgttaaatctctccttgattcaaaaggcattcattacaatcaaggtaacccttacaacctattgacacctgttattgaaaaagtaaaaccaggtgaacaatcttttgtaggtcaacatgcagctacaggatgtgttgctactgcaactgctcaaattatgaaatatcataattaccctaacaaagggttgaaagactacacttacacactaagctcaaataacccatatttcaaccatcctaagaacttgtttgcagctatctctactagacaatacaactggaacaacatcttacctacttatagcggaagagaatctaacgttcaaaaaatggcgatttcagaattgatggctgatgttggtatttcagtagacatggattatggtccatctagtggttctgcaggtagctctcgtgttcaaagagccttgaaagaaaactttggctacaaccaatctgttcaccaaatcaaccgtggcgactttagcaaacaagattgggaagcacaaattgacaaagaattatctcaaaaccaaccagtatactaccaaggtgtcggtaaagtaggcggacatgcctttgttatcgatggtgctgacggacgtaacttctaccatgttaactggggttggggtggagtctctgacggcttcttccgtcttgacgcactaaacccttcagctcttggtactggtggcggcgcaggcggcttcaacggttaccaaagtgctgttgtaggcatcaaaccttag

SEQ ID NO: 2 - protein, 398 aa, Streptococcus pyogenes
MNKKKLGVRLLSLLALGGFVLANPVFADQNFARNEKEAKDSAITFIQKSAAIKAGARSAEDIKLDKVNLGGELSGSNMYVYNISTGGFVIVSGDKRSPEILGYSTSGSFDANGKENIASFMESYVEQIKENKKLDTTYAGTAEIKQPVVKSLLDSKGIHYNQGNPYNLLTPVIEKVKPGEQSFVGQHAATGCVATATAQIMKYHNYPNKGLKDYTYTLSSNNPYFNHPKNLFAAISTRQYNWNNILPTYSGRESNVQKMAISELMADVGISVDMDYGPSSGSAGSSRVQRALKENFGYNQSVHQINRGDFSKQDWEAQIDKELSQNQPVYYQGVGKVGGHAFVIDGADGRNFYHVNWGWGGVSDGFFRLDALNPSALGTGGGAGGFNGYQSAVVGIKP

SEQ ID NO: 3 - DNA, 27 bp, Artificial
  synthetic oligonucleotide
ccatggaaccagttgttaaatctctcc

SEQ ID NO: 4 - DNA, 29 bp, Artificial
  synthetic oligonucleotide
ggatcctaaggtttgatgcctacaacagc

SEQ ID NO: 5 - DNA, 27 bp, Artificial
  synthetic oligonucleotide
ccatggatcaaaactttgctcgtaacg

SEQ ID NO: 6 - DNA, 30 bp, Artificial
  synthetic oligonucleotide
ggatccttatttaatctcagcggtaccagc

SEQ ID NO: 7 - DNA, 23 bp, Artificial
  synthetic oligonucleotide
gctacaggatgtgttgctactgc

SEQ ID NO: 8 - DNA, 23 bp, Artificial
  synthetic oligonucleotide
gcagtagcaacacatcctgtagc

SEQ ID NO: 9 - DNA, 30 bp, Artificial
  synthetic oligonucleotide
agatctaaggagatatacatatggacccag

SEQ ID NO: 10 - DNA, 33 bp, Artificial
  synthetic oligonucleotide
agatctttaagaaggagatatacatatggaacc

SEQ ID NO: 11 - DNA, 39 bp, Artificial
  synthetic oligonucleotide
agatctgcacataactttaagaaggagatatacatatgg

SEQ ID NO: 12 - DNA, 59 bp, Artificial
  synthetic oligonucleotide
agatctaacttgactaaattcgaacagcacataactttaagaaggagatatacatatgg

SEQ ID NO: 13 - DNA, 30 bp, Artificial
  synthetic oligonucleotide
ctcgagctaaggtttgatgcctacaacagc

SEQ ID NO: 14 - DNA, 27 bp, Artificial
  synthetic oligonucleotide
ccatggatcaaaactttgctcgtaacg

SEQ ID NO: 15 - DNA, 30 bp, Artificial; identical to SEQ ID NO: 13
  synthetic oligonucleotide